(19) United States
(12) Patent Application Publication
(10) Pub. No.: US 2022/0249364 A1
(43) Pub. Date: Aug. 11, 2022

(54) Designed Inhibitors of Tight Junction Formation

(71) Applicant: University of Rochester, Rochester, NY (US)

(72) Inventors: Benjamin L. Miller, Rochester, NY (US); Lisa A. Beck, Rochester, NY (US); Matthew G. Brewer, Rochester, NY (US)

(73) Assignee: University of Rochester, Rochester, NY (US)

(21) Appl. No.: 17/596,162

(22) PCT Filed: Jun. 2, 2020
(86) PCT No.: PCT/US20/35658

§ 371 Date: Dec. 3, 2021

Related U.S. Application Data
(60) Provisional application No. 62/857,525, filed on Jun. 5, 2019.

(51) Int. Cl. A61K 9/00; C07K 14/00; A61K 39/145; A61K 39/00; A61K 39/35; A61K 9/70; A61K 47/42; A61P 37/04; A61P 31/16

(52) U.S. Cl. CPC A61K 9/0014; C07K 14/001; A61K 39/145; A61K 39/0011; A61K 38/00; A61K 9/7023; A61K 47/42; A61P 37/04; A61P 31/16; A61K 39/35

(57) ABSTRACT

The present invention relates to isolated peptides suitable for disrupting an epithelial barrier, transepithelial dmg or vaccine formulations, drug delivery vehicles for delivering these formulations, and methods of using of these formulations for disrupting an epithelial barrier.

### CROSS REFERENCE TO RELATED APPLICATION

This application claims priority to U.S. Provisional Application No. 62/857,525 filed on Jun. 5, 2019. The content of the application is incorporated herein by reference in its entirety.

### FIELD OF THE INVENTION

This invention relates to peptides for disrupting an epithelial barrier, transepithelial drug or vaccine formulations, drug delivery vehicles for delivering these formulations, and methods of using of these formulations for disrupting an epithelial or skin barrier.

### BACKGROUND OF THE INVENTION

Intact skin barrier is important for good health, functioning to restrict exposure of environmental toxins, antigens, and pathogens from the immune system (De Benedetto, Kubo, & Beck, 2012; Kubo, Nagao, & Amagai, 2012; O'Neill & Garrod, 2011). As such, it impedes transdermal delivery of therapeutic agents and vaccines. For example, current methods of vaccination primarily rely on intramuscular, subcutaneous, and intradermal injection of antigens. These vaccination routes, while effective, require medical personnel to deliver, generate biohazards (sharps) requiring disposal, and cause patients pain and anxiety. This has fueled research efforts to identify “needle-free” methods of immunization. A number of epicutaneous vaccine delivery systems have been explored, including electroporation and microneedle-based techniques (Leone, Monkare, Bouwstra, & Kersten, 2017; Levin, Kochba, & Kenney, 2014; Todorova et al., 2017). While producing notable successes, each of these methods suffers from complications that make them challenging to implement on a large scale for mass vaccination strategies. Electroporation of antigens into the skin requires expensive machinery and microneedles suffer from inadequate antigen loading, poor reproducibility, incomplete dissolution of microneedles, and costly manufacturing (Ita, 2016). Thus, there is a need for a transepithelial delivery system or formulation to deliver active agents such as vaccines across the skin barrier.

### SUMMARY OF INVENTION

This invention addresses the need mentioned above in a number of aspects.

In one aspect, the invention provides an isolated polypeptide comprising a sequence that is at least 80% (i.e., any number between 80% and 100%, inclusive, e.g., 80%, 85%, 90%, 95%, 99%, and 100%) identical to SEQ ID NO: 3 or 4. In some examples, the polypeptide comprises or consists essentially of SEQ ID NO: 3 or 4. These polypeptides can transiently disrupt tight junctions (TJ) in the epidermis and epithelial barrier function without affecting cell viability. Thus, these tight junction disrupting peptides (TJDPs) can be used to aid transepithelial delivery of active agents.

Accordingly, in a second aspect, the invention provides a transepithelial delivery system or transepithelial delivery composition. The system or composition comprises (i) the polypeptide described above and (ii) a pharmaceutically acceptable carrier. In a preferred embodiment, the transepithelial delivery system or transepithelial delivery composition further comprises (iii) an active agent. (e.g., a therapeutic agent or an antigenic agent). The active agent can be simply mixed with the polypeptide or be conjugated or linked (e.g., via in-frame protein fusion) to the polypeptide.

Accordingly, in some embodiments, the invention provides a therapeutic composition that comprises the transepithelial delivery composition and an effective amount of a therapeutic agent. Examples of the therapeutic agent include a small molecule, a biologic, a nanoparticle, a protein (e.g., an antibody or antigen-binding fragment thereof), a nucleic acid, or a combination thereof (e.g., a gene editing system, such as a CAS-CRISPR system). In other embodiments, the invention provides an immunogenic composition that comprises the transepithelial delivery composition and an effective amount of an antigenic agent. Examples of the antigenic agent include one or more selected from the group consisting of a polysaccharide, a lipid, a protein, a nucleic acid (e.g., one encoding the protein), a small molecule, and a toxin, or an epitope thereof In one embodiment, the antigenic agent can include an antigen of a pathogen or an epitope thereof Examples of the pathogen include a virus, a bacterium, a fungus, and a parasite. Examples of the virus include a picornavirus, a togovirus, a coronavirus, an arenavirus, a bunyavirus, a rhabdovirus, an orthomyxovirus, a paramyxovirus, a reovirus, a parvovirus, a papovovirus, an adenovirus, a herpesvirus, a varicella-zoster virus, and an RNA tumor virus. In one example, the virus is an influenza virus. In another embodiment, the antigenic agent comprises a tumor antigen or an epitope thereof In yet another embodiment, the antigenic agent comprises an allergen or an epitope thereof The above-described transepithelial delivery system, transepithelial delivery composition therapeutic composition, or immunogenic composition can be in the form of a transdermal patch. Example of the coronavirus include severe acute respiratory syndrome coronavirus (SARS-CoV), Middle East Respiratory Syndrome coronavirus (MERS-CoV), and SARS-CoV-2.

The immunogenic composition described above can be used in a method of producing antibodies that recognize an antigen, or eliciting an antigen-specific immune response, in a subject in need thereof To that end, one can administer to the subject the immunogenic composition.

In a third aspect, the invention features an isolated nucleic acid comprising a sequence encoding the polypeptide described above; an expression vector comprising the nucleic acid; and a host cell comprising the nucleic acid. The invention also features a method of producing a polypeptide. The method includes culturing the host cell in a medium under conditions permitting expression of a polypeptide encoded by the nucleic acid, and purifying the polypeptide from the cultured cell or the medium of the cell.

The details of one or more embodiments of the invention are set forth in the description below. Other features, objectives, and advantages of the invention will be apparent from the description and from the claims.

### BRIEF DESCRIPTION OF THE DRAWINGS

FIGS. 1 A and 1B are diagrams showing TJ in the epidermis, Claudin-1 (Cldn1) in TJ, and exemplary peptides used in this study. (A) In the epidermis, TJ (magenta) form a paracellular barrier between keratinocytes in the stratum granulosum (SG). The stratum corneum (SC) and TJ provide barrier function for the skin; L, Langerhans cell (orange); SS, stratum spinosum; SB, stratum basale; BM, basement membrane. (B) Cldn1self-assembles and interacts with TJ proteins through extracellular loops. Peptide 1 (SEQ ID NO: 1) represents half of the first extracellular loop of human Cldn1(light blue), with a Cys to Ser mutation (hCldn1 (53-81, C54, 64S)). Peptide 2 (SEQ ID NO: 2) consists of the same amino acids, but altered sequence order. Peptide 3 and 4 (SEQ ID NOs: 3 and 4) are alterations of 2 that reduce the number of charged residues or remove charge completely, respectively.

FIGS. 2A, 2B, and 2C are diagrams showing that TJDPs decreased barrier function in lung epithelial cells in the absence of cytotoxicity, and enable protein diffusion. (A) Peptides 1 and 2 were used to disrupt 16HBE cells after they had formed TJ. TER was measured daily to observe both disruption and recovery (1 n=3, 2 n=10). (B) Viability changes as a result of peptide exposure were measured at day 1, 2 and 4 (recovery) using the WST-1 assay (1 n=3, 2 n=6).

(C) 24 hours after disruption a monoclonal antibody was applied and diffusion through the monolayer was determined 30 minutes or 18 hours later (n=4). Error bars represent SD. Significance was calculated compared to vehicle control using the Kruskal-Wallis analysis (A/B) and Mann-Whitney t-test (C) within Prism software v8.0. Symbol type signifies peptide concentration that is significant * 50 μM,·10 μM.

FIGS. 3A and 3B are diagrams showing that TJDP delayed barrier formation in primary human foreskin keratinocytes (PHFK) without eliciting cytotoxicity. (A) Peptide 2 was used to disrupt TJ in PHFK during differentiation. Cells were differentiated (media containing 1.8 mM Ca²⁺) in the presence of TJDP for three days after which media was replaced. Transepithelial electrical resistance (TER) was measured over six days to observe disruption and recovery kinetics. Data were normalized to the media controls (n=7-13). (B) Viability changes resulting from Peptide 2 exposure were measured at days 1, 2, and 4 (recovery) using the WST-1 assay (n=5-9). Error bars represent SD. Significance was calculated compared to vehicle control using the Kruskal-Wallis analysis within Prism software v8.0. Symbol type signifies peptide concentration that is significant * 30 μM.

FIGS. 4A and 4B are diagrams showing that TJDP altered staining of TJ proteins (occludin (Ocln) and Cldn1) critical for the establishment of skin barrier function. (A) Cells were exposed to Peptide 2 (10 μM), vehicle or media alone containing Ca²⁺ [1.8 mM] which initiates differentiation. At two and four (recovery) days post differentiation cells were stained for Cldn1 and Ocln (TJ proteins) and nuclei (DAPI). (B) Ten images from each condition were quantified for the number of DAPI+ cells (left), amount of Ocln covered area (center) and level of Cldn1 intensity per cell using ImageJ from a representative donor (n=3). The white bar indicates a 50 um distance. Error bars represent SD. Significance was calculated using the Kruskal-Wallis analysis within Prism software v8.0.

FIG. 5 is a set of diagrams showing that TJDP reduced barrier function of murine skin. 8-10 week old female Balb/c mice were shaved and treated with a depilatory cream. Animals were then rested for three days before TJDP treatment. To disrupt barrier, Peptide 2 (7.8 nmol/cm²) was added to a filter paper (patch) and then applied to mouse skin using a Tegaderm dressing on the right flank (n=10). A vehicle-laden control patch was attached to the left flank of the same animal. 18 hours later the patch was removed and transepithelial water loss (TEWL) was measured 1, 3, and 24 hours later. Lines connect TEWL measurements from a single mouse on either the vehicle or peptide treated flank. Significance was calculated using the paired Wilcoxon t-test within Prism software v8.0.

FIGS. 6A, 6B, 6C, 6D, and 6E are a set of diagrams showing that TJDP could prime and boost the immune system to epicutaneously delivered influenza hemagglutinin. (A) Mice were primed with a patch containing 2 μg HA and either 7.8 or 0.78 nmol/cm² of Peptide 2 or vehicle at day 0, 1, and 2 (n=3). HA was delivered IM as a positive control (n=1). (B) Animals were then boosted intramuscularly with 1 μg of inactivated influenza virus 21 days later. (C) Animals were primed IM and then patch boosted (n=4) or IM injected (n=3) as a positive control. (D) Anti-HA serum antibodies were measured prior to and after boost. (E) HAI titers were measured to determine whether protective antibodies were elicited. Error bars represent SD. Significance was calculated using the Mann-Whitney t-test within Prism software v8.0.

FIG. 7 is a table showing range of TER values from untreated 16HBE and PHFK cells used in FIGS. 2 and 3.

FIGS. 8A and 8B show an immunofluorescence microscopy of TJ proteins in 16HBE cells. Untreated 16HBE cells stained for: (A) Cldn1(green) and zona occluders-1 (ZO-1, red); (B) Cldn4 (green) and Ocln (red). The white bar indicates a 25 μm distance.

FIGS. 9A and 9B show that TJDP decreased barrier function in lung epithelial cells with varying efficiency. (A) Peptide 3 and (B) 4 (derived from the Cldn1sequence) were used to disrupt 16HBE cells after they had formed TJ. Cells were exposed to multiple concentrations of TJDP for two days and then new media was added. TER was measured over the course of four days to observe both disruption and recovery kinetics. Data were normalized to the media control (n=3). Error bars represent SD. Significance was calculated compared to vehicle control using the Kruskal-Wallis analysis within Prism software v8.0. Symbol type signifies peptide concentration that is significant * 30 μM,·10 μM.

FIG. 10 shows that TJDP had specificity for disrupting barrier. Peptide 1, 2 and a (FKFE)₂ peptide were used to disrupt 16HBE cells after they had formed TJ. Cells were exposed to different concentrations of peptide (12 or 96 μM) and TER was measured over the course of 24 hours to observe the kinetics of disruption. Data is presented as average ohms/cm² measured (n=2).

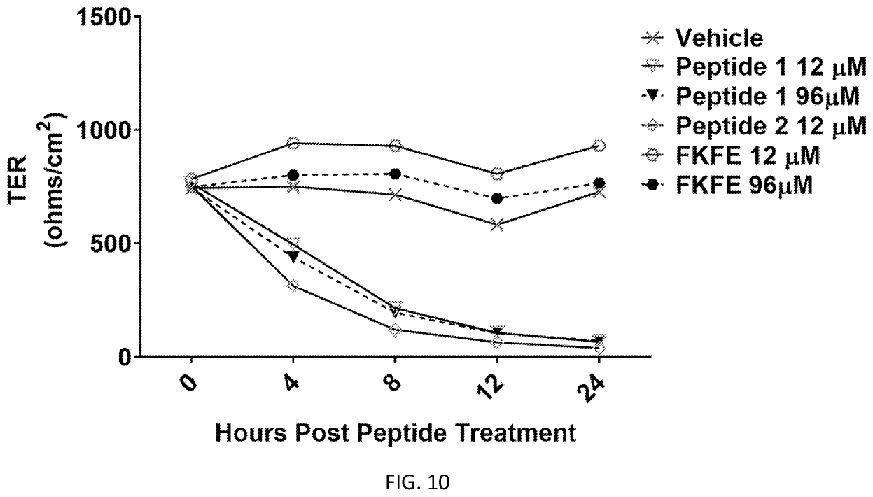

FIG. 11 shows that TJDP altered staining of TJ proteins (Ocln and Cldn1) critical for the establishment of skin barrier function. Representative images are higher magnification pictures of the images from FIG. 4 to better show distribution of TJ proteins. The white bar indicates a 25 μm distance.

FIGS. 12A, 12B, 12C, and 12D shows that change in TEWL after mice were treated with either a patch containing peptide 2 or vehicle. Shown are the raw TEWL values in grams/hour/meter² from FIG. 5. The grey line indicates the baseline average of both sites on the mouse (TEWL of ˜9 g/h/m²).

### DETAILED DESCRIPTION OF THE INVENTION

This invention is based, at least in part, on an unexpected discovery of various mutant TJDP polypeptides. These polypeptides and related compositions are useful for disrupting an epithelial barrier and delivering various active agents cross the epithelial and skin barrier.

### Skin Barrier and Tight Junctions

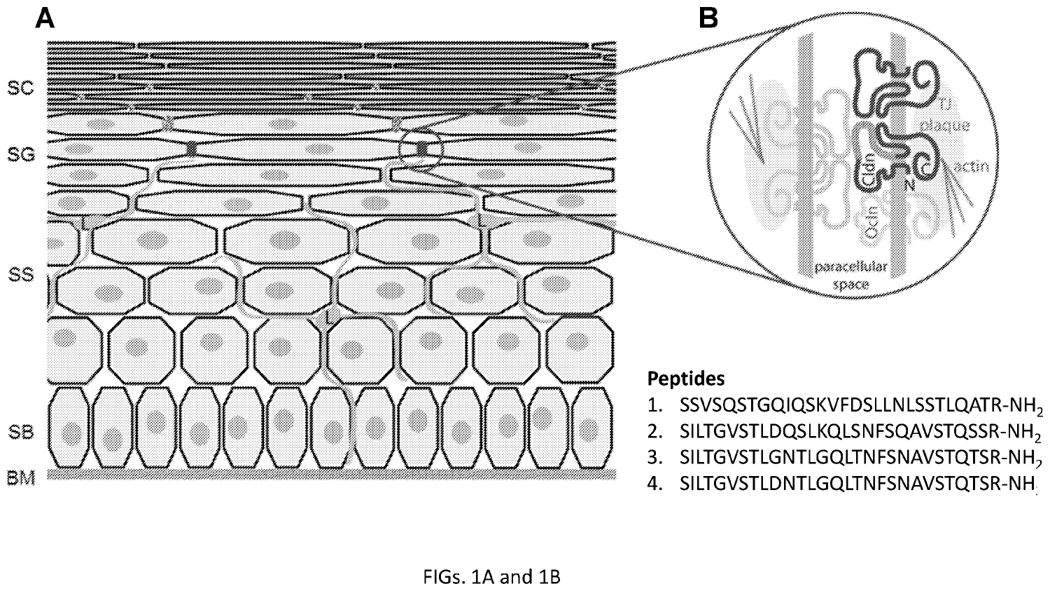

In the skin, tight junctions (TJ) and the stratum corneum (SC) act together to maintain a formidable epidermal barrier (FIG. 1). TJ are composed of claudin proteins that control barrier formation by homodimerizing on adjacent cells through extracellular loop domain interaction (Haftek et al., 2011; Sugawara et al., 2013; Yoshida et al., 2013). The composition of these extracellular loops determine the claudin function, ranging from a tight seal to a more “leaky” channel (Gunzel, 2017). Disruption of TJ and specifically, reduced expression of Cldn1, is a key feature of human and canine atopic dermatitis (De Benedetto, Rafaels, et al., 2011; De Benedetto, Slifka, et al., 2011; Roussel, Bruet, Marsella, Knol, & Bourdeau, 2015; Tokumasu, Tamura, & Tsukita, 2017). TJ disruption results in increased movement of molecules and viruses via the paracellular route both into and out of the lower levels of the epidermis (De Benedetto, Slifka, et al., 2011). This process can be measured by TEWL or tracer flux through the epidermis, both of which are increased when TJ are disrupted (Furuse et al., 2002). Cldn1knockout mice have extensive TEWL, indicating that the protein is essential for skin barrier integrity (Furuse et al., 2002). A less dramatic variation of this is seen in humans with atopic dermatitis (AD): these patients have been shown to have greater TEWL and paracellular permeability than healthy subjects. This is thought to be due to reductions in Cldn1, with expression levels ˜50% lower than in non-atopic controls (De Benedetto, Rafaels, et al., 2011). Therefore, by targeting Cldn1, one can disrupt TJ function and in so doing enhance paracellular permeability and facilitate greater epicutaneous adaptive immune reactivity. Importantly, it was shown that synthetic peptides derived from the sequence of the extracellular loops of transmembrane TJ proteins, Cldns and Ocln, are able to disrupt barrier function at high concentrations in addition to causing mislocalization of Ocln (Baumgartner, Beeman, Hodges, & Neville, 2011; Beeman, Webb, & Baumgartner, 2012; Mrsny et al., 2008; Wong & Gumbiner, 1997; Zwanziger, Hackel, et al., 2012; Zwanziger, Staat, Andjelkovic, & Blasig, 2012).

Keratinocytes express many pattern recognition receptors (PRRs) that enhance the skin's adaptive immune response to epicutaneous antigens. These PRRs are expressed below TJ, strongly implicating TJ disruption as a critical step in antigen responsiveness.

As disclosed herein, to disrupt TJ, inventors designed peptides inspired by the first extracellular loop of the TJ transmembrane protein, claudin-1. These peptides transiently disrupted TJ in the human lung epithelial cell line 16HBE, and delayed TJ formation in primary human keratinocytes. Building on these observations, inventors tested whether vaccinating mice with an epicutaneous influenza patch containing TJ-disrupting peptides was an effective strategy to elicit an immunogenic response. Application of a TJDP patch resulted in barrier disruption as measured by increased transepithelial water loss. Invention observed a significant increase in antigen-specific antibodies when they applied patches with TJDP plus antigen (e.g., influenza hemagglutinin) in either a patch-prime or a patch-boost model. Collectively, these observations demonstrate that the designed peptides perturb TJ in human lung as well as human and murine skin epithelium enabling epicutaneous vaccine delivery. This approach can obviate currently used needle-based vaccination methods that require administration by health care workers and biohazard waste removal.

As disclosed herein, inventors synthesized a number of peptides including:

| Name | Sequences | SEQ ID NO |
|---|---|---|
| Peptide 1 | SSVSQSTGQIQSKVFDSLLNLSSTLQATR | 1 |
| Peptide 2 | SILTGVSTLDQSLKQLSNFSQAVSTQSSR | 2 |
| Peptide 3 | SILTGVSTLGNTLGQLTNFSNAVSTQTSR | 3 |
| Peptide 4 | SILTGVSTLDNTLGQLTNFSNAVSTQTSR | 4 |

One of them, Peptide 1, is derived from amino acid residues 53-81 of the first extracellular loop of human Cldn1, which has extensive homology with mouse Cldn1, containing only one amino acid change (S->N at position 74). This domain was chosen as a target for TJ disruption since the first extracellular loop has been shown to facilitate transepithelial electrical resistance development and determine ion permeability selectivity. As a test of the importance of sequence order vs. overall amino acid identity, inventors also synthesized a peptide, called Peptide 2, with the same amino acid composition as Peptide 1, but with altered sequence. Additional Peptides 3 and 4 (FIG. 1) were designed to test if reducing the number of charged residues while retaining the net charge of the peptide (Peptide 3) or reducing the net charge to zero (Peptide 4) diminished the ability to disrupt TJ.

When all four of these peptides were used in an epithelial cell model derived from human lung cells (16HBE) they showed robust TJ disruption. Peptide 2, which showed the most robust TJ disruption in 16HBE cells, also was able to significantly delay TJ formation in primary human foreskin keratinocytes (PHFK). To extend these findings an in vivo patch-based delivery system was developed to determine if Peptide 2 could enhance an immune response against the viral protein, influenza hemagglutinin (HA), in a mouse vaccination model. Perturbed skin barrier was observed by increased TEWL after application of a patch containing TJDP. Furthermore, serological studies indicated that antigens delivered in tandem with a TJDP had an enhanced humoral immune response. Overall, this work establishes the validity of using TJ disruption as a method to deliver antigens epicutaneously, suggesting that this approach may be useful as a vaccine or drug delivery method.

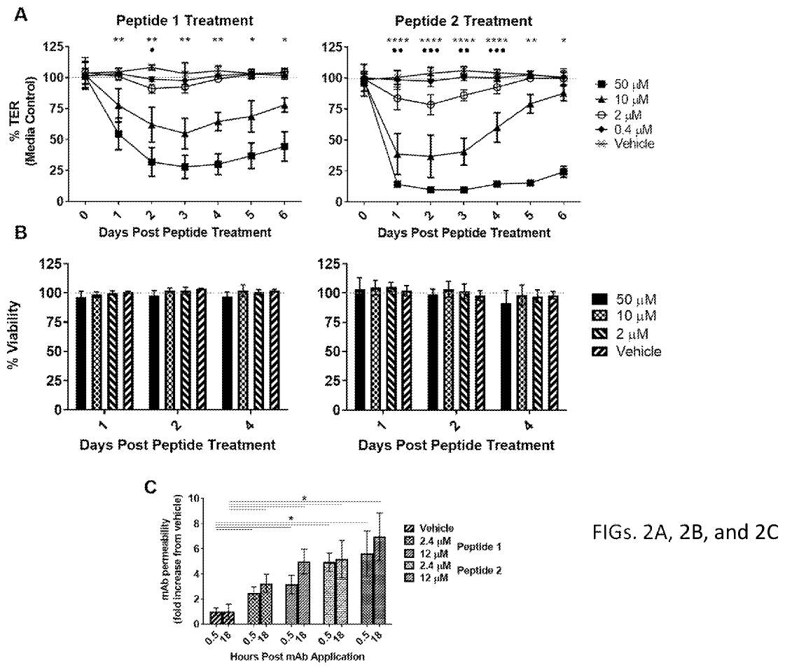

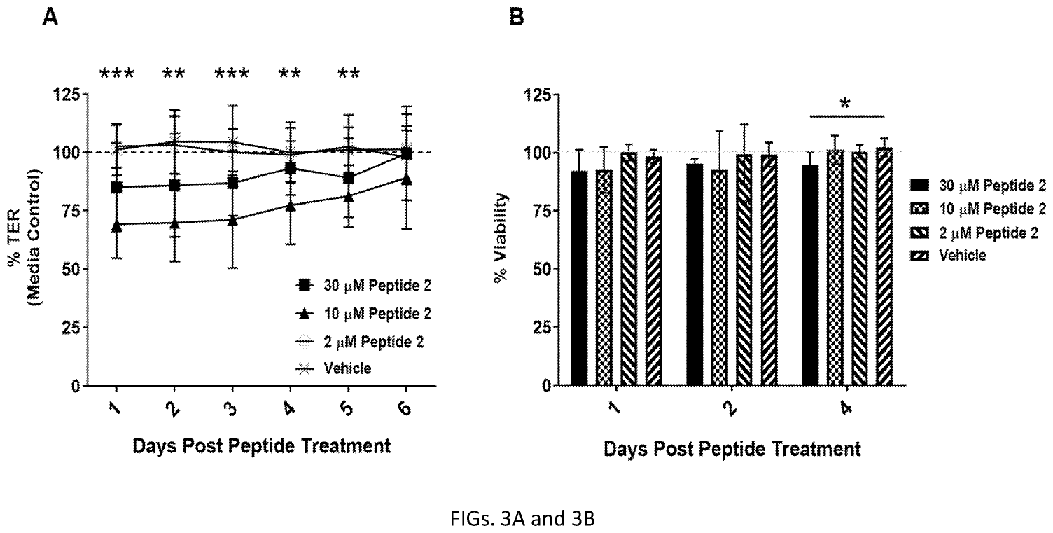

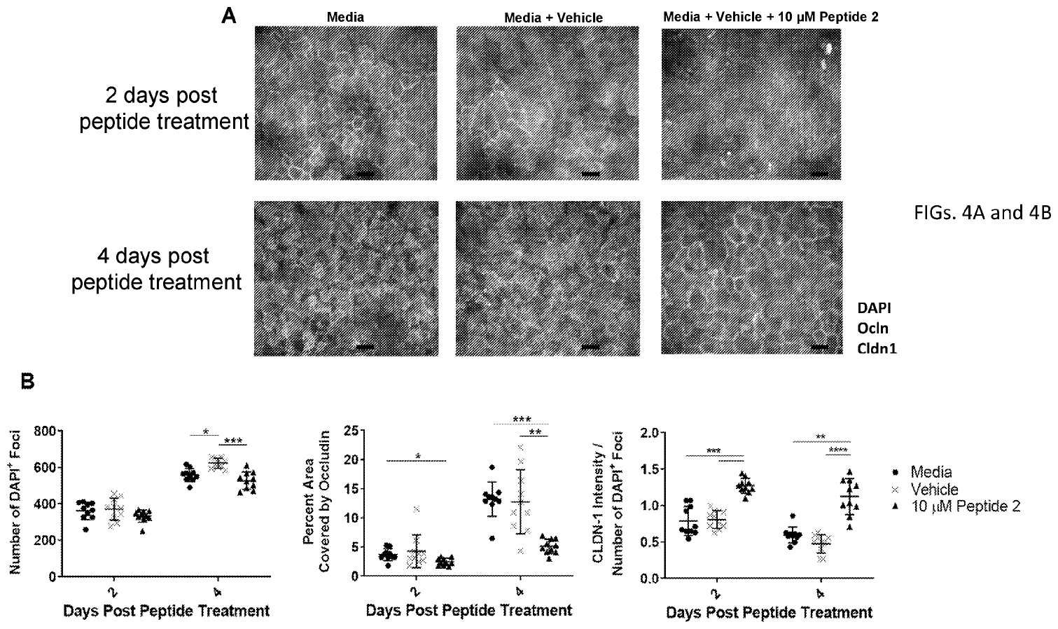

The results disclosed herein suggest an alternative method of vaccine delivery. The method disclosed here, TJ disruption, avoids all complications of needle-based delivery since a patch-based delivery is painless, can be dried (avoids refrigeration) and is easily applied. Inventors have demonstrated that TJDP based on the first loop of Cldn1 (FIG. 1) disrupt barrier function in a lung epithelial cell line, in the absence of cytotoxicity, and this disruption is significant enough to allow the diffusion of large molecular weight proteins (150 kDa) (FIG. 2). Using primary epidermal cells, TJDP were able to delay barrier formation without impacting cell viability (FIG. 3). To further characterize this barrier disruption, PHFK monolayers were treated with Peptide 2 and visualized for TJ protein immunoreactivity during differentiation. In peptide-treated PHFK, Ocln staining was substantially delayed and Cldn1 staining was mainly detectable in the cytoplasm (FIG. 4). These observations suggest that TJDP perturb barrier function, at least in part, by altering the expression and/or localization of key TJ transmembrane proteins.

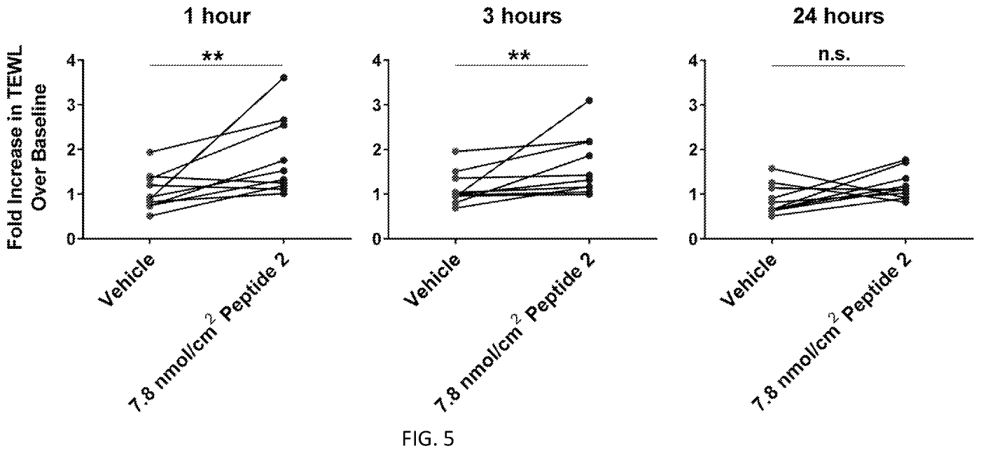

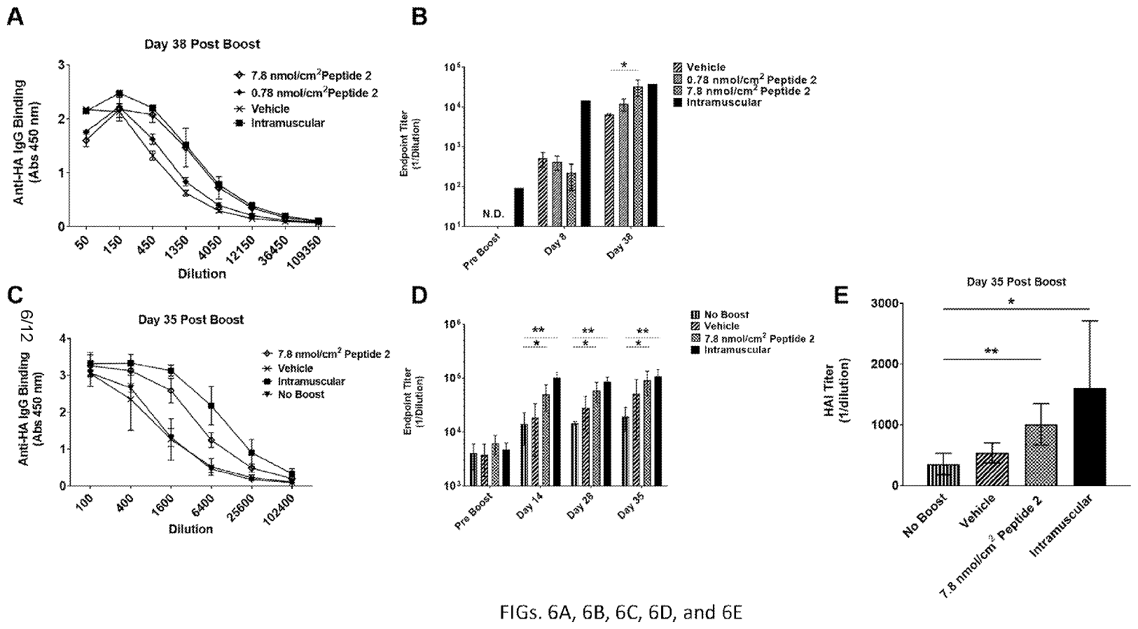

Murine studies disclosed herein confirmed that TJDP do in fact disrupt the skin barrier, as measured by increased TEWL. Importantly, this effect was transient, with TEWL recovering to near baseline values within 24 hours (FIG. 5). To determine the biological consequences of epidermal disruption as a non-invasive vaccination method, inventors tested whether a patch with a viral antigen and the TJDP could (1) prime the naive immune system and/or (2) boost pre-existing immunity to a protein. Studies aimed at priming the naive immune system to HA antigen established memory as was observed by enhanced antibody responses after IM boost. Even vehicle delivery of protein (in a patch) elicited a boost response, suggesting that skin occlusion is sufficient to deliver an antigen to the murine immune system, even in the context of minimal changes in TEWL (FIGS. 5 and 6A). Importantly, in all of their mouse studies, inventors observed no physical changes in the skin over the 3-month period the mice were observed. This observation suggests that TJ-disruption in mouse skin does not promote a disease state and/or increase skin infection risks, highlighting the safety of this transepidermal antigen delivery system.

Humans are exposed to influenza as young as 6 months of age and as a result most individuals have preexisting immunity to the virus (Zhou et al., 2012). Therefore, the function of seasonal flu shots is to stimulate the expansion of influenza-specific, memory B cells to the likely seasonal strains. To model whether an epicutaneous patch with a TJDP could boost the immune response to influenza, an IM injection of inactivated virus was used to establish a memory or “pre-existing immunity” state. To boost, HA was then delivered by a patch containing a TJDP to skin or by IM injection as positive control. Animals receiving a patch containing TJDP and antigen were observed to have enhanced levels of antigen specific antibodies similar to the IM control (FIGS. 6C-D). Importantly, patch-based delivery of HA stimulated increased HAI titers, which are a known correlate of protection against influenza (Plotkin, 2010). This observation suggests that TJ disruption-based antigen delivery through the skin can elicit antibodies that are biologically significant in protection from influenza.

The data presented here demonstrate that a TJDP disclosed herein transiently disrupts epithelial barrier function at doses that do not affect viability. Their incorporation into an epicutaneous patch provides a non-invasive, painless method to administer vaccines quickly and cheaply to a large population. Importantly, multiple groups are attempting to establish a universal flu vaccine that would increase the effectiveness of the current vaccine and possibly negate yearly booster immunizations (Erbelding et al., 2018; Nachbagauer et al., 2017). One method to accomplish this is to stimulate cytotoxic CD8⁺ T cells specific for conserved epitopes in the virus. Typical IM vaccination is extremely poor at eliciting cellular immunity, but skin-based delivery of antigen has been shown to initiate robust T cell responses that home to other organs in the body (Liu, Fuhlbrigge, Karibian, Tian, & Kupper, 2006; Schmidt et al., 2016; Zaric et al., 2017). Therefore, methods described in this invention can be used to initiate a universal response to pathogens, such as influenza, addressing important public health concerns.

### Peptides

In certain aspects, this invention provides an agent that transiently disrupts claudin-1 within TJs. The agent includes a peptide described herein. In one embodiment, the peptide can have low solubility or be insoluble in aqueous media in the absence of surfactant. The peptide may associate with or bind to native claudin-1 within the TJ. In one embodiment, the peptide of this invention herein has an amino acid sequence that is not naturally occurring in claudin-1.

The peptide comprises a sequence that is at least 80% (e.g., any number between 80% and 100%, inclusive, e.g., 80%, 85%, 90%, 95%, 96%, 97%, 98%, 99%, and 100%) identical to SEQ

ID NO: 3 or 4. The amino acid sequence of the peptide may include an amino acid sequence of at least 5 (e.g., 6, 7, 8, 9, 10, 11, 12, 13, 14, 15, 16, 17, 18, 19, 20, 21, 22, 23, 24, 25, 26, 27, 28, 29, or 30) amino acid residues. For example, the peptide can be 5 to 500, 10 to 100, or 15-50 (e.g., 10 to 70, 20 to 50, 20-35, and 25 to 30) amino acid residues.

The amino acid composition of the above-mentioned peptide or variant thereof may vary without disrupting the ability to disrupt an epithelial barrier. For example, it can contain one or more conservative amino acid modifications or substitutions. As used herein, the term “conservative sequence modifications” refers to amino acid modifications that do not significantly affect or alter the characteristics of the peptide having the amino acid sequence of SEQ ID NO: 3 or 4. Conservative amino acid substitutions are ones in which an amino acid residue is replaced with an amino acid residue having a similar side chain. Families of amino acid residues having similar side chains are known and have been defined in the art.

Amino acid substitutions can be made, in some cases, by selecting substitutions that do not differ significantly in their effect on maintaining (a) the structure of the peptide backbone in the area of the substitution, (b) the charge or hydrophobicity of the molecule at the target sit; or (c) the bulk of the side chain. For example, naturally occurring residues can be divided into groups based on side-chain properties; (1) hydrophobic amino acids (norleucine, methionine, alanine, valine, leucine, and isoleucine); (2) neutral hydrophilic amino acids (cysteine, serine, threonine, asparagine, and glutamine,); (3) acidic amino acids (aspartic acid and glutamic acid); (4) basic amino acids (histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Examples of substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. Exemplary substitutions are shown in the table below.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Thus, a predicted nonessential amino acid residue in SEQ ID NO: 3 or 4 can be replaced with another amino acid residue from the same side chain family. Alternatively, mutations can be introduced randomly along all or part of the sequences, such as by saturation mutagenesis, and the resultant mutants can be screened for the ability to disrupt TJ as described below or in, e.g., WO2015/024022 and U.S. Pat. No. 9,757,428, the contents of which are incorporated by reference in their entireties.

The peptides described herein can be presented in the form of a fusion peptide that includes, in addition, a second amino acid sequence coupled to the peptides via peptide bond. The second amino acid sequence can be an active agent, which are discussed below. Alternatively, the second amino acid sequence can be a purification tag, such as poly-histidine (His6), a glutathione-S-transferase (GST-), or maltose-binding protein (MBP-), which assists in the purification but can later be removed, i.e., cleaved from the peptide following recovery. Protease-specific cleavage sites (i.e., in a cleavable linker sequence) can be introduced between the purification tag and the desired peptide. The desired peptide product can be purified further to remove the cleaved purification tags.

According to one approach, the peptides described herein can be synthesized by standard peptide synthesis operations. These can include both FMOC (9-fluorenylmethyloxy-carbonyl) and tBoc (tert-butyloxy-carbonyl) synthesis protocols that can be carried out on automated solid phase peptide synthesis instruments including, without limitation, the Applied Biosystems 431 A, 433 A synthesizers and Peptide Technologies Symphony or large scale Sonata or CEM Liberty automated solid phase peptide synthesizers. The use of alternative peptide synthesis instruments is also contemplated. Peptides prepared using solid phase synthesis can be recovered in a substantially pure form.

The peptides described herein may be also prepared by using recombinant expression systems followed by separation and purification of the recombinantly prepared peptides. Generally, this involves inserting an encoding nucleic acid molecule into an expression system or vector to which the molecule is heterologous (i.e., not normally present). One or more desired nucleic acid molecules encoding a peptide described herein may be inserted into the vector. The heterologous nucleic acid molecule can be inserted into the expression system or vector in proper sense (5′-3′) orientation and correct reading frame relative to a promoter and any other 5′ and 3′ regulatory elements.

Nucleic acid molecules encoding the peptides described herein can be prepared via solid-phase synthesis using, e.g., the phosphoramidite method and phosphoramidite building blocks derived from protected 2′-deoxynucleosides. To obtain the desired oligonucleotide, the building blocks can be sequentially coupled to the growing oligonucleotide chain in the order required by the sequence of the product. Upon the completion of the chain assembly, the product be released from the solid phase to solution, deprotected, collected, and typically purified using HPLC. The limits of solid phase synthesis are suitable for preparing oligonucleotides up to about 200 nt in length, which encodes peptides on the order of about 65 amino acids or less. The ends of the synthetized oligonucleotide can be designed to include specific restriction enzyme cleavage site to facilitate ligation of the synthesized oligonucleotide into an expression vector.

For longer peptides, oligonucleotides can be prepared via solid phase synthesis and then the synthetic oligonucleotide sequences ligated together using various techniques. Recombinant techniques for the fabrication of whole synthetic genes are reviewed, for example, in Hughes et al., “Chapter Twelve--Gene Synthesis: Methods and Applications,” Methods in Enzymology 498:277-309 (2011), which is hereby incorporated by reference in its entirety.

Once a suitable expression vector is selected, the desired nucleic acid sequences can be cloned into the vector using standard cloning procedures in the art, as described by Sambrook et al., Molecular Cloning: A Laboratory Manual, Cold Springs Laboratory, Cold Springs Harbor, N.Y. (1989), or U.S. Pat. No. 4,237,224 to Cohen and Boyer, which are hereby incorporated by reference in their entirety. The vector can be then introduced to a suitable host.

A variety of host-vector systems may be utilized to recombinantly express the peptides described herein. Primarily, the vector system must be compatible with the host used. Host-vector systems include, without limitation, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used to carry out this and other aspects described herein.

When it is desirable to achieve heterologous expression of a peptide, DNA molecules encoding the peptide can be delivered into the cell. This includes providing a nucleic acid molecule encoding the desired product, and then introducing the nucleic acid molecule into the cell under conditions effective to express the desired product in the cell. Preferably, this is achieved by inserting the nucleic acid molecule into an expression vector before it is introduced into the cell.

Purified peptides may be obtained by several methods. The peptide may be produced in purified form (preferably at least about 80% or 85% pure, or at least about 90% or 95% pure) by conventional techniques. Depending on whether the recombinant host cell is made to secrete the peptide into growth medium (see U.S. Pat. No. 6,596,509, which is hereby incorporated by reference in its entirety), the peptide can be isolated and purified by centrifugation (to separate cellular components from supernatant containing the secreted peptide) followed by sequential ammonium sulfate precipitation of the supernatant. The fraction containing the peptide can be subjected to gel filtration in an appropriately sized dextran or polyacrylamide column to separate the peptides from other proteins. If necessary, the peptide fraction may be further purified by HPLC.

Alternatively, if the peptide is not secreted, it can be isolated from the recombinant cells using standard isolation and purification schemes. This includes disrupting the cells (e.g., by sonication, freezing, French press, etc.) and then recovering the peptide from the cellular debris.

Purification can be achieved using the centrifugation, precipitation, and purification procedures described above. The use of purification tags, described above, can simplify this process. Once the peptides described herein are recovered, they can be used to prepare a composition as described herein.

### Transepithelial Delivery Compositions and Systems

The peptides described above can alter the permeability an epithelial barrier. Accordingly, they are useful in a composition or system for transepithelial delivery of an agent, such as a drug or a vaccine formulation. The term epithelia is used in its usual sense and relates to the epithelium, the outside layer of cells that covers all the free, open surfaces of the body including cutaneous (skin) and mucous membranes. The term transepithelial refers to entry of a substance such as a drug, vaccine, or active agent through the epithelium, including direct topical application and application using a support material such as a patch. Peptides and compositions described herein are also useful in altering the permeability of blood vessels and blood brain barrier.

### Active Agents

Regardless of the embodiment, active agents described herein can be administered via pharmaceutical composition or formulation. Accordingly, within scope of this invention are pharmaceutical compositions or formulations including one or more peptides described above, a pharmaceutically acceptable carrier, and an active agent. Peptides described herein may be present in an amount suitable to disrupt TJ function in epithelial cells. For instance, the peptide may be present in an amount by weight of about 0.000001 to about 25%. The peptide may be present at a concentration of less than about 500 μM (e.g., less than about 400, 300, 200, 100, 50, 40, 30, 20, 10, 5, 2, and 1 μM).

As used herein, the term “active agent” means an agent that is intended to have an effect on an individual. Active agents include, without limitation, therapeutic agents that are intended for use in the diagnosis, cure, treatment, or prevention of disease. The term “drug” and “therapeutic agent” are used interchangeably and are intended to have their broadest interpretation as any therapeutically active substance which is delivered to a living organism to produce a desired, usually beneficial, effect. In general, this includes therapeutic agents in all of therapeutic areas including, but not limited to, biologics (e.g., growth factors, cytokines, and antibodies), nucleic acids (e.g., DNA, RNA, and derivatives thereof) antiinfectives, antibiotics, antiviral agents, analgesics, fentanyl, sufentanil, buprenorphine, analgesic combinations, anesthetics, anorexics, antiarthritics, antiasthmatic agents, terbutaline, anticonvulsants, antidepressants, antidiabetic agents, antidiarrheals, antihistamines, antiinflammatory agents, antimigraine preparations, antimotion sickness, scopolamine, ondansetron, antinauseants, antineoplastics, antiparkinsonism drugs, cardiostimulants, dobutamine, antipruritics, antipsychotics, antipyretics, antispasmodics, gastrointestinal and urinary, anticholinergics, sympathomimetics, xanthine derivatives, cardiovascular preparations, calcium channel blockers, nifedipine, beta-blockers, beta-agonists, salbutamol, ritodrine, antiarrythmics, antihypertensives, atenolol, ACE inhibitors, diuretics, vasodilators, coronary, peripheral and cerebral, central nervous system stimulants, cough and cold preparations, decongestants, diagnostics, hormones, parathyroid hormone, growth hormone, insulin, hypnotics, immunosuppressives, muscle relaxants, parasympatholytics, parasympathomimetics, anti-oxidants, nicotine, prostaglandins, psychostimulants, sedatives, tranquilizers, skin acting anti-oxidants, caretenoids, ascorbic acid (vitamin C), vitamin E, anti wrinkling agents, retinoids, retinol (vitamin A alcohol), alpha-hydroxic acids, beta-hydroxy acid, salicylic acid, combination-hydroxy acids and poly-hydroxy acids, and hydrolyzed and soluble collagen, hyaluronic acid, anticellulite agents, aminophyllines, skin bleaching agents, retinoic acid, hydroquinone, peroxides, botanical preparations or extracts, and combinations thereof

Additional active agents include one or more antigenic agents that are present in a vaccine composition. Antigenic agents may include proteins or polypeptides, nucleic acids, lipids, carbohydrates, lipopolysaccharides, etc., which are intended to induce an immune response against a pathogen, infected cell, or cell characterized by a disease state (e.g., cancerous cell).

### Immunogenic Compositions

In one aspect illustrated herein, the pharmaceutical composition of this invention is an immunogenic composition, such as a vaccine. The vaccine can be a transepithelial vaccine formulation that would benefit from TJ disruption at the site of vaccine delivery. The transepithelial vaccine formulation may be a formulation suitable for administration to any epithelial site, including cutaneous (e.g., transdermal formulation) and mucous membranes. In one embodiment, the transepithelial vaccine formulation is a transdermal vaccine formulation. The transdermal vaccine can be in the form of a patch worn by the user, whereby moisture from the vaccine recipient's body allows for delivery of the active agents across the skin (i.e., at the site of application).

The transepithelial vaccine formulations of aspects illustrated herein may include a pharmaceutically suitable carrier, an effective amount of an antigen or antigen-encoding nucleic acid molecule present in the carrier, optionally one or more adjuvants, and an agent that transiently disrupts claudin-1 function within tight junctions according to aspects illustrated herein. The formulation is presented in the transepithelial delivery vehicle, as is known in the art.

Vaccination at, for example, the epidermal surface may be accomplished by targeting Langerhan cells in the epidermis with agents according to aspects illustrated herein. Similar strategies have been used to target M cells in mucosal surfaces with claudin-4 specific peptides (Lo et al., “M Cell Targeting by a Claudin 4 Targeting Peptide Can Enhance Mucosal IgA Responses,” BMC Biotech. 12:7 (2012), which is hereby incorporated by reference in its entirety).

Any suitable antigen or antigen-encoding nucleic acid molecule, or a combination thereof, can be used in the vaccine formulations of aspects illustrated herein. Exemplary classes of vaccine antigen include, without limitation, an allergen, an immunogenic subunit derived from a pathogen, a virus-like particle, an attenuated virus particle, or glycoprotein or glycolipid conjugated to an immunogenic polypeptide. Antigen-encoding nucleic acid molecules can be in the form of naked DNA or expression vectors, as well as infective transformation vectors. In certain embodiments, the antigen (e.g., allergen) cab be coupled to mixed with an adjuvant.

A number of known transepithelial vaccine formulations can be modified to include an agent that alters TJ barrier function in epithelial cells. One exemplary transdermal vaccine formulation that can be modified is described in U.S. Pat. No. 6,420,176, which is hereby incorporated by reference in its entirety. For example, the carrier may comprise one or more of sugar, polylysine, polyethylenimine, polyethylenimine derivatives, and liposomes, together with their derivatives. One preferred carrier of this type is a mannosylated polyethylenimine. The DermaVir transdermal delivery system is believed to employ these types of carriers.

Another exemplary transdermal vaccine formulation that can be modified is described in U.S. Pat. No. 6,869,607 to Buschle et al., which is hereby incorporated by reference in its entirety. For example, the carrier may comprise a solution or emulsion that is substantially free of inorganic salt ions and includes one or more water soluble or water-emulsifiable substances capable of making the vaccine isotonic or hypotonic (e.g., maltose, fructose, galactose, saccharose, sugar alcohol, lipid; or combinations thereof), and an adjuvant that is a polycation (e.g., polylysine or polyarginine) optionally modified with a sugar group. The adjuvant, according to one embodiment, can be a combination of a polycation and an immunostimulatory CpG or non-CpG oligodeoxynucleotide. One form of this adjuvant is the Intercell adjuvant IC31. Yet another exemplary vaccine formulation that can be modified is described in U.S. Pat. No. 7,247,433 to Rose, which is hereby incorporated by reference in its entirety. For example, HPV virus-like particles could be administered with a pharmaceutically acceptable carrier and with or without E. coli LT R192G as the adjuvant.

As noted above, formulations (including vaccine formulations) according to aspects illustrated herein may be delivered via aspiration, airway instillation, aerosolization, nebulization, intranasal instillation, oral or nasogastic instillation, intraperitoneal injection, or intravascular injection. Pulmonary delivery of vaccine formulations according to aspects illustrated herein may be carried out according to techniques known to those of skill in the art (see, e.g., Lu et al., “Pulmonary Vaccine Delivery,” Expert Rev. Vaccines 6(2): 213-226 (2007), which is hereby incorporated by reference in its entirety). An exemplary vaccine formulation that can be modified is described in US2013/0183336, which is hereby incorporated by reference in its entirety. Suitable devices for delivering vaccine formulations according to aspects illustrated herein include, for example, nebulizers (see, e.g., US 2013/0032140, which is hereby incorporated by reference in its entirely).

As noted herein, such vaccine formulations illustrated herein may include surfactants. In addition to those noted above, suitable surfactants for use in accordance with aspects illustrated herein include those that are suitable for use in vaccine formulations suitable for pulmonary delivery (see, e.g., Lu et al., “Pulmonary Vaccine Delivery,” Expert Rev. Vaccines 6(2): 213-226 (2007), WO 2013/120058, and WO 2008/011559, which are hereby incorporated by reference in their entirety).

The skilled artisan will understand that there are no limitations on the identities of the antigenic components in an immunogenic composition of the present invention. The immunogenic composition discussed herein can be designed to contain any antigenic agent, antigen, immunogen, or epitope of interest. The antigen may contain a protein, a polypeptide, a peptide, an epitope, a hapten, or any combination thereof The antigen can also contain a whole organism, killed, attenuated or live; a subunit or portion of an organism; a recombinant vector containing an insert with immunogenic properties; a piece or fragment of DNA capable of inducing an immune response upon presentation to a host animal. Alternately, the immunogen or antigen may contain a toxin or antitoxin.

In certain embodiments the antigenic component can come from a disease-causing microorganism. For example, it can be antigen or epitope from a virus of any one of the virus families: Adenoviridae (e.g., Adenovirus, infectious canine hepatitis virus), Papovaviridae (e.g., Papillomavirus, polyomaviridae, simian vacuolating virus), Parvoviridae (e.g., Parvovirus B19, canine parvovirus), Herpesviridae (e.g., Herpes simplex virus, varicella-zoster virus, cytomegalovirus, Epstein-Barr virus), Poxviridae (e.g., Smallpox virus, cow pox virus, sheep pox virus, orf virus, monkey pox virus, vaccinia virus), Hepadnaviridae (e.g., Hepatitis B virus),

Anelloviridae (e.g., Torque teno virus), Reoviridae (e.g., Reovirus, rotavirus), Picornaviridae (e.g., Enterovirus, rhinovirus, hepatovirus, cardiovirus, aphthovirus, poliovirus, parechovirus, erbovirus, kobuvirus, teschovirus, coxsackie), Caliciviridae (e.g., Norwalk virus), Togaviridae (e.g., Rubella virus, alphavirus), Arenaviridae (e.g., Lymphocytic choriomeningitis virus), Flaviviridae (e.g., Dengue virus, hepatitis C virus, yellow fever virus), Orthomyxoviridae (e.g., Influenzavirus A, influenzavirus B, influenzavirus C, isavirus, thogotovirus), Paramyxoviridae (e.g., Measles virus, mumps virus, respiratory syncytial virus, Rinderpest virus, canine distemper virus), Bunyaviridae (e.g., California encephalitis virus, hantavirus), Rhabdoviridae (e.g., Rabies virus), Filoviridae (e.g., Ebola virus, Marburg virus), Coronaviridae (e.g., Corona virus), Astroviridae (e.g., Astrovirus), Bornaviridae (e.g., Borna disease virus), Arteriviridae (e.g., Arterivirus, equine arteritis virus), and Hepeviridae (e.g., Hepatitis E virus).

In one example, the antigen can be a HA protein derived from an influenza virus to elicit flu immunity, especially pan-flu immunity. Other influenza epitopes or proteins, such as the neuraminidase (NA), can be used to elicit immunity to various distinct influenza types or other epitopes of viral origin.

Additional examples of suitable antigens, epitopes, or immunogenic moieties include prion, bacterial, or parasitic antigens; inactivated viral, tumor-derived, protozoal, organism-derived, fungal, or bacterial antigens; toxoids, toxins; self-antigens; food allergens (peanut, etc.); pertussis antigens (e.g., detoxified pertussis toxin) polysaccharides; lipids, fatty acids, proteins; glycoproteins; peptides; cellular vaccines; DNA vaccines; recombinant proteins; glycoproteins; and the like. These antigens and related immunogenic/vaccine compositions can be used for eliciting immune response to, for example, MMR (measles, mumps, and rubella), Tdap (tetanus-diphtheria-acelluar pertussis), hepatitis A, hepatitis B, hepatitis C, Dengue, Ebola, HPV, Varicella, Haemophilus influenza type B, Japanese Encephalitis, Meningococcal, Pneumococal, Polio, Rabies, Shingles—Herpes Zoster, Whooping cough, Yellow fever, BCG, cholera, plague, typhoid, influenza A, influenza B, parainfluenza, polio, rabies, measles, mumps, rubella, tetanus, diphtheria, hemophilus tuberculosis, meningococcal and pneumococcal vaccines, adenovirus, HIV, chicken pox, cytomegalovirus, feline leukemia, fowl plague, HSV-1 and HSV-2, hog cholera, respiratory syncytial virus, rotavirus, papilloma virus and yellow fever, and Alzheimer's Disease. Especially, materials (such as recombinant proteins, glycoproteins, peptides, and haptens) that otherwise do not raise a strong immune response can be used in connection with the invention so as to elicit satisfactory response.

In some embodiments, the epitope can be a portion of a cancer antigen, such that antibodies against the epitope can raise specific anti-cancer immunity. This will be particularly interesting in situations where passive infusion of specific antibodies is known to be therapeutic (as is the case with neurofibromatosis, a childhood cancer), or where specific anti-tumor antibodies can bind to receptors present in certain cancer tissues (e.g., breast) and inhibit cancer growth (e.g. Trastuzumab/herceptin, broadly used in breast cancer treatment to block neu/her receptors).

The terms cancer antigen and tumor antigen are used interchangeably and refer to an antigen that is differentially expressed by cancer cells. Cancer antigens can be exploited to differentially target an immune response against cancer cells, and stimulate tumor-specific immune responses. Certain cancer antigens are encoded, though not necessarily expressed, by normal cells. Some of these antigens may be characterized as normally silent (i.e., not expressed) in normal cells, those that are expressed only at certain stages of differentiation, and those that are temporally expressed (e.g., embryonic and fetal antigens). Other cancer antigens can be encoded by mutant cellular genes such as, for example, oncogenes (e.g., activated ras oncogene), suppressor genes (e.g., mutant p53), or fusion proteins resulting from internal deletions or chromosomal translocations. Still other cancer antigens can be encoded by viral genes such as those carried by RNA and DNA tumor viruses.

Examples of tumor antigens include MAGE, MART-1/Melan-A, gp100, Dipeptidyl peptidase IV (DPPUV), adenosine deaminase-binding protein (ADAbp), cyclophilin b, Colorectal associated antigen (CRC)-0017-1A/GA733, Carcinoembryonic Antigen (CEA) and its antigenic epitopes CAP-1 and CAP-2, etv6, am11, Prostate Specific Antigen (PSA) and its antigenic epitopes PSA-1, PSA-2, and PSA-3, prostate-specific membrane antigen (PSMA), T-cell receptor/CD3-ζ chain, MAGE-family of tumor antigens (e.g., MAGE-A1 MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p2lras, RCAS1, a-fetoprotein, E-cadherin, α-catenin, β-catenin, γ-catenin, p120ctn, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-3, SSX-4, SSX-5, SCP-1 and CT-7, and c-erbB-2.

Cancers or tumors and specific tumor antigens associated with such tumors (but not exclusively), include acute lymphoblastic leukemia (etv6, am11, cyclophilin b), B cell lymphoma (Ig-idiotype), glioma (E-cadherin, α-catenin, (β-catenin, γ-catenin, and p120ctn), bladder cancer (p2lras), biliary cancer (p2lras), breast cancer (MUC family, HER2/neu, c-erbB-2), cervical carcinoma (p53, p2lras), colon carcinoma (p2lras, HER2/neu, c-erbB-2, MUC family), colorectal cancer (Colorectal associated antigen (CRC)-CO17-1A/GA733, APC), choriocarcinoma (CEA), epithelial cell cancer (cyclophilin b), gastric cancer (HER2/neu, c-erbB-2, ga733 glycoprotein), hepatocellular cancer (.alpha.-fetoprotein), Hodgkins lymphoma (Imp-1, EBNA-1), lung cancer (CEA, MAGE-3, NY-ESO-1), lymphoid cell-derived leukemia (cyclophilin b), melanoma (p5 protein, gp75, oncofetal antigen, GM2 and GD2 gangliosides, Melan-A/MART-1, cdc27, MAGE-3, p2lras, gp100), myeloma (MUC family, p2lras), non-small cell lung carcinoma (HER2/neu, c-erbB-2), nasopharyngeal cancer (Imp-1, EBNA-1), ovarian cancer (MUC family, HER2/neu, c-erbB-2), prostate cancer (Prostate Specific Antigen (PSA) and its antigenic epitopes PSA-1, PSA-2, and PSA-3, PSMA, HER2/neu, c-erbB-2, ga733 glycoprotein), renal cancer (HER2/neu, c-erbB-2), squamous cell cancers of the cervix and esophagus (viral products such as human papilloma virus proteins), testicular cancer (NY-ESO-1), and T cell leukemia (HTLV-1 epitopes).

Each of the above-described polypeptide/protein components of the immunogenic composition can be obtained as a recombinant polypeptide/protein. To prepare a recombinant polypeptide, a nucleic acid encoding it (e.g., SEQ ID NO: 3 or 4) can be linked to another nucleic acid encoding a fusion partner, e.g., GST, 6x-His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this invention. Alternatively, the peptides/polypeptides/proteins of the invention can be chemically synthesized (see e.g., Creighton, “Proteins: Structures and Molecular Principles,” W. H. Freeman & Co., N.Y., 1983), or produced by recombinant DNA technology as described herein. For additional guidance, skilled artisans may consult Ausubel et al. (supra), Sambrook et al. (“Molecular Cloning, A Laboratory Manual,” Cold Spring Harbor Press, Cold Spring Harbor, N.Y., 1989), and, particularly for examples of chemical synthesis Gait, M. J. Ed. (“Oligonucleotide Synthesis,” IRL Press, Oxford, 1984).

The peptide/polypeptide/protein of this invention covers chemically modified versions. Examples of chemically modified peptide/protein include those subjected to conformational change, addition or deletion of a sugar chain, and those to which a compound such as polyethylene glycol has been bound. Once purified and tested by standard methods or according to the methods described in the examples below, the peptide/polypeptide/protein can be included in a pharmaceutical composition.

The immunogenic composition of the invention may be used to immunize an animal. An immunogenic composition according to the invention is preferably used for the preparation of a vaccine. Preferably a prophylactic and/or therapeutic vaccine is produced. Thus, within the scope of this invention is an immunogenic or vaccine composition that contains a pharmaceutically acceptable carrier, an effective amount of a peptide described above, and an effective amount of an antigenic agent. The carriers used in the composition can be selected on the basis of the mode and route of administration, and standard pharmaceutical practice.

The composition can contain an adjuvant. Examples of an adjuvant include a cholera toxin, Escherichia coli heat-labile enterotoxin, liposome, unmethylated DNA (CpG) or any other innate immune-stimulating complex. Various adjuvants that can be used to further increase the immunological response depend on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

A vaccine formulation may be administered to a subject per se or in the form of a pharmaceutical or therapeutic composition. Pharmaceutical compositions containing a peptide of the invention and an adjuvant may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the antigens of the invention into preparations which can be used pharmaceutically. Proper formulation is dependent upon the route of administration chosen.

For injection, vaccine preparations may be formulated in aqueous solutions, preferably in physiologically compatible buffers such as Hanks' solution, Ringer's solution, phosphate buffered saline, or any other physiological saline buffer. The solution may contain formulatory agents such as suspending, stabilizing and/or dispersing agents. Alternatively, the immunogenic composition described above may be in powder form for constitution with a suitable vehicle, e.g., sterile pyrogen-free water, before use.

The amount of a composition administered depends, for example, on the particular antigen in the composition, whether an adjuvant is co-administered with the antigen, the type of adjuvant co-administered, the mode and frequency of administration, and the desired effect (e.g., protection or treatment), as can be determined by one skilled in the art. Determination of an effective amount of the vaccine formulation for administration is well within the capabilities of those skilled in the art, especially in light of the detailed disclosure provided herein. An effective dose can be estimated initially from in vitro assays. For example, a dose can be formulated in animal models to achieve an induction of an immune response using techniques that are well known in the art. One having ordinary skill in the art could readily optimize administration to all animal species based on results described herein. Dosage amount and interval may be adjusted individually. For example, when used as a vaccine, the vaccine formulations of the invention may be administered in about 1 to 3 doses for a 1-36 week period. Preferably, 1 or 2 doses are administered, at intervals of about 3 weeks to about 4 months, and booster vaccinations may be given periodically thereafter. Alternative protocols may be appropriate for individual animals. A suitable dose is an amount of the vaccine formulation that, when administered as described above, is capable of raising an immune response in an immunized animal sufficient to protect the animal from an infection for at least 4 to 12 months. In general, the amount of the antigen present in a dose ranges from about 1 pg to about 100 mg per kg of host, typically from about 10 pg to about 1 mg, and preferably from about 100 pg to about 1 pg. Suitable dose range will vary with the route of injection and the size of the subject, but will typically range from about 0.1 mL to about 5 mL. Sera can be taken from the subject for testing the immune response or antibody production elicited by the composition against the antigen. Methods of assaying antibodies against a specific antigen are well known in the art. Additional boosters can be given as needed. By varying the amount of the composition and frequency of administration, the protocol can be optimized for eliciting a maximal production of the antibodies.

### Therapeutic Compositions

Alternatively, the pharmaceutical composition is a transepithelial (e.g., transdermal or transmucosal) therapeutic or drug formulation. The drug formulation includes a pharmaceutically acceptable carrier, an effective amount of a therapeutic agent, and an agent that transiently disrupts claudin-1 within tight junctions.

The term “pharmaceutically acceptable carrier” refers to any suitable adjuvants, carriers, excipients, or stabilizers, and can be in solid or liquid form such as tablets, capsules, powders, solutions, suspensions, or emulsions. In certain embodiments according to aspects illustrated herein, the carrier may be in the form of a lotion, cream, gel, emulsion, ointment, solution, suspension, foam, or paste.

In one embodiment, the carrier includes an oil-in water emulsion. In one embodiment, the carrier includes tromethane ethanol, polyethylene glycol, glycerin, propylene glycol, acrylates, Carbopol, purified water, benzyl alcohol, cetyl alcohol, citric acid, monoglycerides, diglycerides, triglycerides, oleyl alcohol, sodium cetostearylsulphate, sodium hydroxide, stearyl alcohol, white petrolatum, mineral oil, propylene carbonate, white wax, paraffin, or any combination thereof

Compositions and/or carriers described herein may also be in the form of aqueous solutions that include a surfactant, particularly when the agents that alter TJ barrier function are insoluble or only partially soluble in the aqueous carriers. Suitable surfactants include, for example, nonionic surfactant polyols. In one embodiment, the surfactant is PLURONIC F-127. Other known surfactant or solubilizer additives may be used. Examples include, but are not limited to, solubilizers like TWEEN 20 (polyoxyethylene (20) sorbitan monolaurate), TWEEN 40 (polyoxyethylene (20) sorbitan monopalmitate), TWEEN 80 (polyoxyethylene (20) sorbitan monooleate), PLURONIC F-127, PLURONIC F-68 (polyoxyethylene polyoxypropylene block copolymers), PEG (polyethylene glycol), non-ionic surfactants such as polysorbate 20 or 80 or poloxamer 184 or 188, PLURONIC polyls, other block co-polymers, and chelators such as EDTA and EGTA.

Compositions described herein may also include lung surfactant formulations tailored for delivery to the lung epithelium. For instance, suitable formulations that may be modified for use in accordance with aspects illustrated herein include those described in WO2015024022, WO 2013/120058, and WO 2008/011559, which are hereby incorporated by reference in their entirety. Such compositions may readily form liposomal vesicles that can be used to deliver all classes of agents described herein to a patient. The administration of such compositions can be any suitable approach for delivery of the therapeutic agent to a target tissue, including aspiration, airway instillation, aerosolization, nebulization, intranasal instillation, oral or nasogastic instillation, intraperitoneal injection, or intravascular injection. The target tissue can be lung tissue or a systemic tissue. The agent or agents to be delivered can be any pharmaceutical or therapeutic agent including those described herein.

Surfactants and/or additives described herein may be used alone or in combination in amounts by weight of, for example, about 0.001 to about 5.0%, about 0.001 to about 4.0%, or about 0.001 to about 3.0%. In one embodiment, the composition comprises about 0.12% surfactant (e.g., PLURONIC F-127). In one embodiment, the composition comprises about 0.006% surfactant (e.g., PLURONIC F-127).

Compositions described herein may include a suitable carrier, as described above. The pharmaceutical compositions may be formulated for administrating topically (as described above with respect to transepithelial, transdermal or transmucosal formulations) or by any other means suitable. For example, the compositions may be formulated for administration orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes. The formulations may conveniently be presented in unit dosage form and may be prepared by any of the methods well known in the art of pharmacy. Carrier(s) may be present in an amount by weight of, for example, about 10 to about 99%, about 20 to about 99%, about 30 to about 99%, about 40 to about 99%, about 50 to about 99%, about 60 to about 99%, about 70 to about 99%, about 80 to about 99%, about 90 to about 99%.

Compositions described herein include a peptide as described herein along with one or more of a pharmaceutically acceptable carrier, surfactant, and optionally one or more therapeutic agents, as described above. For example, the carrier may be present in the amount of 40-99% by weight, the surfactant may be present in an amount of up to 5% by weight of the composition, and the peptide may be present in an amount of about 0.000001 to about 25% by weight of the composition.

Typically, a composition will contain from about 0.01 to about 90 percent (e.g., up to about 1, 5, 10, 20, 30, 40, 50, 60, 70, 80, or 90 percent) by weight of active agent(s)), together with the adjuvants, carriers, and/or excipients. For instance, the therapeutic agent may present in an amount by weight of about 0.01 to about 90% (e.g., 0.01 to about 80%, about 0.01 to about 10%, about 0.1 to about 80%, about 0.1 to about 50%, about 0.1 to about 10%, or about 0.1 to about 5%).

While individual needs may vary, determination of optimal ranges of effective amounts of each component is within the skill of the art. Typical dosages of the therapeutic agent comprise about 0.01 to about 100 mg/kg body wt. Other dosages may comprise about 0.1 to about 100 mg/kg body wt. or about 1 to about 100 mg/kg body wt. Treatment regimen for the administration of the agents can also be determined readily by those with ordinary skill in art. That is, the frequency of administration and size of the dose can be established by routine optimization, preferably while minimizing any side effects.

Compositions and/or carriers according to aspects illustrated herein may include an artificial vesicle. The artificial vesicle may be any suitable artificial vesicle known to those of skill in the art. In certain embodiments according to aspects illustrated herein, the artificial vesicle may be a microparticle, nanoparticle, or the like. Such will be known to those of skill in the art and may include any suitable materials (e.g., BSA, polymer microgels silica). In one embodiment, the artificial vesicle is a liposome or a micelle.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), each of which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane, which enzyme slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in its entirety. Like liposomes, micelles have also been used in the art for drug delivery. A number of different micelle formulations have been described in the literature for use in delivery proteins or polypeptides, and others have been described which are suitable for delivery of nucleic acids. Any suitable micelle formulations can be adapted for delivery of the therapeutic protein or polypeptide or nucleic acids aspects illustrated herein. Exemplary micelles include without limitation those described, e.g., in U.S. Pat. No. 6,210,717 to Choi et al.; and U.S. Pat. No. 6,835,718 to Kosak, each of which is hereby incorporated by reference in its entirety.

Aspects illustrated herein are also useful in the controlled delivery of polypeptide and protein drugs and other macromolecular drugs. These macromolecular substances typically have a molecular weight of at least about 300 daltons, and more typically a molecular weight in the range of about 300 to 40,000 daltons. In one embodiment, the therapeutic is at least 300 daltons in size.

In another embodiment, the therapeutic is at least 500 daltons in size. In yet a further embodiment, the therapeutic is not less than 300 daltons in size.

Specific examples of peptides, proteins, and macromolecules in this size range include, without limitation, LHRH, LHRH analogs such as buserelin, gonadorelin, napharelin and leuprolide, GHRH, GHRF, insulin, insulotropin, heparin, calcitonin, octreotide, endorphin, TRH, NT-36 (chemical name: N=[[(s)-4-oxo-2-azetidinyl]carbonyl]-L-histidyl-L-prolinamide), liprecin, pituitary hormones (e.g., HGH, HMG, HCG, desmopressin acetate, etc.), follicle luteoids, .alpha.ANF, growth factors such as growth factor releasing factor (GFRF), .beta.MSH, somatostatin, atrial natriuretic peptide, bradykinin, somatotropin, platelet-derived growth factor, asparaginase, bleomycin sulfate, chymopapain, cholecystokinin, chorionic gonadotropin, corticotropin (ACTH), epidermal growth factor, erythropoietin, epoprostenol (platelet aggregation inhibitor), follicle stimulating hormone, glucagon, hirulog, and other analogs of hirudin, hyaluronidase, interferon, insulin-like growth factors, interleukin-1, interleukin-2, menotropins (urofollitropin (FSH) and LH), oxytocin, streptokinase, tissue plasminogen activator, urokinase, vasopressin, desmopressin, ACTH analogs, ANP, ANP clearance inhibitors, angiotensin II antagonists, antidiuretic hormone agonists, antidiuretic hormone antagonists, bradykinin antagonists, CD4, ceredase, CSF's, enkephalins, FAB fragments, IgE peptide suppressors, IGF-1, neuropeptide Y, neurotrophic factors, oligodeoxynucleotides and their analogues such as antisense RNA, antisense DNA and anti-gene nucleic acids, opiate peptides, colony stimulating factors, parathyroid hormone and agonists, parathyroid hormone antagonists, prostaglandin antagonists, pentigetide, protein C, protein S, ramoplanin, renin inhibitors, thymosin alpha-1, thrombolytics, TNF, vaccines, vasopressin antagonist analogs, alpha-1 anti-trypsin (recombinant), and TGF-beta.

### Systems

Another aspect of this invention relates to a transdermal delivery device or patch. The transdermal drug delivery device includes an agent or a transdermal vaccine or drug formulation according to aspects illustrated herein. In one embodiment, the transdermal patch includes a backing material, an adhesive material in contact with a first portion of the backing material; and a drug storage material comprising the agent or transdermal vaccine or drug formulation, where the drug storage material is in contact with a second portion of the backing material. In one embodiment the patch also includes a releasable liner material to be removed upon application to the skin.

Any suitable backing material known in the art of transdermal patches (such as a breathable material) may be used in accordance with aspects illustrated herein. The backing is flexible such that the device conforms to the skin. Exemplary backing materials include conventional flexible backing materials used for pressure sensitive tapes, such as polyethylene, particularly low density polyethylene, linear low density polyethylene, high density polyethylene, polyester, polyethylene terephthalate, randomly oriented nylon fibers, polypropylene, ethylene-vinyl acetate copolymer, polyurethane, rayon and the like. Backings that are layered, such as polyethylene-aluminum-polyethylene composites, are also suitable. The backing should be substantially inert to the ingredients of the drug storage material.

Adhesives suitable for use with aspects illustrated herein with any dermatologically acceptable adhesive. Examples of dermatologically acceptable adhesives include, but are not limited to acrylics, natural and synthetic rubbers, ethylene vinyl acetate, poly(alpha-olefins), vinyl ethers, silicones, copolymers thereof and mixtures thereof In an embodiment, the first adhesive layer includes a silicone adhesive (e.g., BIO-PSA 7-4302 Silicone Adhesive available commercially from DOW CORNING).

The transdermal patch may optionally include one or more release liners for storage or handling purposes. Many suitable release liners are known within the art. The release liner can be made of a polymeric material that may be optionally metallized. Examples of suitable polymeric materials include, but are not limited to, polyurethane, polyvinyl acetate, polyvinylidene chloride, polypropylene, polycarbonate, polystyrene, polyethylene, polyethylene terephthalate (PET), polybutylene terephthalate, paper, and combinations thereof In certain embodiments, the release liner is siliconized. In other embodiments, the release liner is coated with fluoropolymer, such as PET coated with fluoropolymer (e.g., SCOTCHPAK 9744 from 3M).

The drug storage material may be any dermatologically acceptable material suitable for use as a drug storage material or reservoir in a transdermal patch. For instance, the drug storage material may be a polymer. Examples of polymers include microporous polyolefin film (e.g., SOLUPOR from SOLUTECH), acrylonitrile films, polyethylnapthalene, polyethylene terephthalate (PET), polyimide, polyurethane, polyethylene, polypropylene, ethylene-vinyl acetate (EVA), copolymers thereof and mixtures thereof In one embodiment, the polymer is EVA. In another embodiment, the polymer is EVA having a vinyl acetate content by weight in the range of about 4% to about 19%. In a preferred embodiment, the polymer is EVA having vinyl acetate content by weight of about 9%. The drug storage material may also include a heat-sealable material for attaching to other components. As an example, the heat-sealable permeable layer may be an EVA membrane, such as COTRAN 9702, available commercially from 3M.

Other delivery devices including compositions according to aspects illustrated herein are also contemplated. Such devices include those suitable for delivery of compositions according to aspects illustrated herein via aspiration, airway instillation, aerosolization, nebulization, intranasal instillation, oral or nasogastic instillation, intraperitoneal injection, or intravascular injection. Exemplary devices include inhalers or nebulizers (see, e.g., US2013/0032140, which is hereby incorporated by reference in its entirely).

### Uses

This invention provides a method of disrupting an epithelial barrier. The method involves applying to an epithelial site an amount of a peptide describe above that is effective to disrupt claudin-1 in keratinocytes present at the site, thereby disrupting barrier formation at the epithelial site. Also contemplated are methods of disrupting an epithelial barrier by applying to an epithelial site a pharmaceutical composition described herein, thereby disrupting barrier formation at the epithelial site.

Accordingly, a further aspect of this invention relates to a method of administering a pharmaceutical composition (e.g., an immunogenic/vaccine composition or a drug formulation) to a subject. The method involves applying the transepithelial an immunogenic/vaccine formulation to an epithelial site on the subject. The region of epithelia (e.g., skin) to be treated in accordance with aspects illustrated herein is dependent on the intended purpose for delivery. For instance, for transdermal drug or vaccine delivery, the drug or vaccine may be administered to a region of the skin such as the upper arm, back, or the like. The drug or vaccine may also be administered via other routes as described herein.

The immunogenic composition disclosed herein can be used as an antibody-stimulating platform, to raise antibodies against any antigenic agent, antigen, immunogen, or epitope of interest. The immunogenic composition of the invention can therefore be used as a prophylactic vaccine and therapeutic vaccine for treating various conditions. The composition can be administered as the single therapeutic agent in a treatment regimen. Alternatively, it can be administered in combination with another therapeutic composition, or with other active agents such as antivirals, antibiotics, etc. In particular, the composition of this invention can be useful for treating viral diseases and tumors. This immunomodulation activity suggests that the immunogenic or vaccine composition of the invention is useful in treating conditions such as, but not limited to:

(a) viral diseases such as diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such as chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection; and

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers.

### Additional Definitions

The terms “peptide,” “polypeptide,” and “protein” are used herein interchangeably to describe the arrangement of amino acid residues in a polymer. A peptide, polypeptide, or protein can be composed of the standard 20 naturally occurring amino acid, in addition to rare amino acids and synthetic amino acid analogs. They can be any chain of amino acids, regardless of length or post-translational modification (for example, glycosylation or phosphorylation).

A “recombinant” peptide, polypeptide, or protein refers to a peptide, polypeptide, or protein produced by recombinant DNA techniques; i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof It retains substantially the activity to of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent (e.g., one of SEQ ID NOs: 1-4). Accordingly, within scope of this invention are hinge regions having one or more point mutations, insertions, deletions, truncations, a fusion protein, or a combination thereof.

As used herein, the percent homology between two amino acid sequences is equivalent to the percent identity between the two sequences. The percent identity between the two sequences is a function of the number of identical positions shared by the sequences (i.e., % homology=# of identical positions/total # of positions x 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

An “isolated” peptide, polypeptide, or protein refers to a peptide, polypeptide, or protein that has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide/protein described in the invention can be purified from a natural source, produced by recombinant DNA techniques, or by chemical methods.

As used herein, “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

“Antigenic agent,” “antigen,” or “immunogen” means a substance that induces a specific immune response in a host animal. It can be a molecule containing one or more epitopes (either linear, conformational or both) that elicit an immunological response. The term “epitope” refers to basic element or smallest unit of recognition by an individual antibody, B-cell receptor, or T-cell receptor, and thus the particular domain, region or molecular structure to which said antibody or T-cell receptor binds. An antigen may consist of numerous epitopes while a hapten, typically, may possess few epitopes.

The term “immunogenic” refers to a capability of producing an immune response in a host animal against an antigen or antigens. This immune response forms the basis of the protective immunity elicited by a vaccine against a specific infectious organism. “Immune response” refers to a response elicited in an animal, which may refer to cellular immunity (CMI); humoral immunity or both.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

As used herein, a “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental, non-human animal or animal suitable as a disease model. The term “animal” includes all vertebrate animals including humans. In particular, the term “vertebrate animal” includes, but not limited to, humans, canines (e.g., dogs), felines (e.g., cats); equines (e.g., horses), bovines (e.g., cattle), porcine (e.g., pigs), as well as in avians.

As used herein, “treating” or “treatment” (e.g., a viral infection, tumor or cancer) refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder. When the terms “prevent”, “preventing”, and “prevention” are used herein in connection with a given treatment for a given condition, they mean that the treated patient either does not develop a clinically observable level of the condition at all, or develops it more slowly and/or to a lesser degree than he/she would have absent the treatment. These terms are not limited solely to a situation in which the patient experiences no aspect of the condition whatsoever. For example, a treatment will be said to have “prevented” the condition if it is given during exposure of a patient to a stimulus that would have been expected to produce a given manifestation of the condition, and results in the patient's experiencing fewer and/or milder symptoms of the condition than otherwise expected. For example, a treatment can “prevent” infection by resulting the patient's displaying only mild overt symptoms of the infection; it does not imply that there must have been no penetration of any cell by the infecting microorganism.

An effective amount refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment. For example, a therapeutically effective amount to treat or inhibit a viral infection is an amount that will cause a reduction in one or more of the manifestations of viral infection, such as viral lesions, viral load, rate of virus production, and mortality as compared to untreated control animals. Similarly, a therapeutically effective amount of a combination to treat a neoplastic condition is an amount that will cause, for example, a reduction in tumor size, a reduction in the number of tumor foci, or slow the growth of a tumor, as compared to untreated animals.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

### EXAMPLES

### Example 1

This example descibes material and methods used in Examples 2-6 bellow.

### Primary Human Foreskin Keratinocytes (PHFK) and Human Bronchial Epithelial (16HBE) Cell Culture

PHFK were isolated from discarded foreskin tissue. Isolation and propagation procedures for both PHFK and 16HBE cells were done as previously described (Poumay, Roland, Leclercq-Smekens, & Leloup, 1994; Saatian et al., 2013).

### Tight Junction Disrupting Peptide Formulation

10 mgs of synthesized TJDP (RS SYNTHESIS) was dissolved in 100 μl of DMSO (SIGMA). Dissolved peptide was then diluted in pre-warmed (55 ° C.) DMSO/PLURONIC F127® (SPECTRUM) solution formulated in PBS (0.6% / 0.12%, respectively). This solution was heated at 55° C. for 30 minutes then homogenized into detergent using a water bath sonicator (BRANSON 2200) for 10 minutes and vortexed for 1 minute. Peptide was stored at 4° C. until use.

TER measurements and Paracellular Flux

TER and paracellular flux were done as previously published (De Benedetto, Rafaels, et al., 2011). Briefly, measurements of TER were taken for up to 6 days following exposure to TJ disrupting peptides or vehicle. 2 μM of fluorescently-labeled antibody (palivizumab) was added to cells after 24 hours of exposure to TJDP and paracellular flux was measured 30 minutes and 18 hours later.

### Cell Viability Measurement

For cytotoxicity measurements, cells were plated at a density of 75,000 cells/well in a 96-well plate and grown to confluence (2 days). Cells were then exposed to vehicle or TJDP and viability was measured at 24, 48 and 96 hours. WST-1 reagent (ROCHE) was diluted 20-fold into each well, and cells were incubated at 37° C. Duplicate readings were taken at 0.5 and 1 hour after addition using a THERMO MULTISKAN EX plate reader (A450—background A620). Media only wells were subtracted and values were normalized to media treated controls.

### Immunoflourescent Staining of TJ Formation in PHFK

150,000 PHFK or 16HBE cells were plated onto glass coverslips. Cells were grown to confluence over three days and treated with TJDP (10 μM), vehicle (0.0015% DMSO/0.0003% PLURONIC F-127) or media alone for 48 and 96 hours. Cells were fixed in 4% paraformaldehyde for 10 minutes and washed three times in PBS. Following this, cells were permeabilized with 100% ice cold methanol for 15 minutes at −20° C. then washed in PBS three times and left overnight at 4° C. The next day, cells were blocked in 1% BSA dissolved in PBS for one hour and stained with anti-Cldn1and anti-Ocln (INVITROGEN, 500- or 300-fold dilution, respectively) for 2.5 hours at RT. Primary antibodies were detected with anti-mouse ALEXA FLUOR568 and anti-rabbit ALEXA FLUOR488 (LIFE TECHNOLOGIES) and nuclei with 4′,6-diamidino-2-phenylindole (INVITROGEN) all at a 1000-fold dilution. Coverslips were mounted onto glass slides with 15 μl of PROLONG GOLD ANTIFADE REAGENT (INVITROGEN).

Quantification of TJ Protein Levels and DAPI Staining by Fluorescence Microscopy PHFK and 16HBE slides were imaged on an OLYMPUS BX60 fluorescent microscope equipped with SPOT RT3 (DIAGNOSTIC INSTRUMENTS, INC.). Images were processed with IMAGEJ software. To quantify DAPI⁺ foci, background was first removed using the threshold function and then converted to black/white using the binary tool. Individual cells were highlighted with the watershed function. Nuclei sized pixels were then counted with the analyze particles function set to a pixel² range of 500-Infinity to exclude signals too small to be nuclei. To quantify Cldn1intensity image was processed as above. The measure function was then used to give a mean and standard deviation of Cldn1intensity. To account for variation in the monolayer each image was divided into quadrants using the rectangle tool analyzed to confirm homogeneity across a single image. To quantify area covered by Ocln the image was processed as above. Oc1n signal was selected using the create selection tool and Ocln positive area was measured with the analyze (measure) function. The entire area of the image was measured using the same process without creating a selection and used to calculate area of a single image covered by Ocln. Composite images were generated with IMAGEJ software. Background fluorescence was minimized with the threshold function to enhance signal-to-noise and DAPI, Cldn1, and Oc1n channels were overlayed and pseudocolored blue, green and red respectively.

### Patch Treatment and TEWL Measurements of Mouse Skin

All animal studies were approved by the University of Rochester's committee on animal resources (protocol 2017-017) in accordance with the NIH Guide for the Care and Use of Laboratory Animals. Female Balb/c mice (8-10 weeks old) were anesthetized with an intraperitoneal injection of ketamine (100 mg/kg -MYLAN) /xylazine (20 mg/kg solution ANASED) in saline solution (HOSPIRA). Hair was removed from both flanks by shaving (Oster) and application of a depilatory cream (VEET). Animals were rested for three days and then anesthetized again for patch application. Patches were created using a square 0.64 cm² piece of filter paper (IQ CHAMBER) applied to a 2 cm ² piece of TEGADERM dressing (3M). TJDP (7.8 or 0.78 nmol/cm²) or vehicle were then applied to filter paper and allowed to absorb before application. Peptide treated patches were affixed to the mouse's right flank, while vehicle treated patches were placed on the left flank. After 18 hours, patches were removed and the skin was allowed to dry before TEWL was measured at 1, 3, and 24 hours post-patch removal using a TEWAMETER TM 300.

### Animal Immunizations

Mice received either 2 μg of recombinant A/Cal/07/2009 hemagglutinin (provided by Dr. Florian Krammer, Mount Sinai, NY) or 1 μg of beta propiolactone inactivated influenza virus. Immunization was delivered either by patch application as stated above or a 50 μl IM injection into the flank muscle. Animals were boosted either by patch or injection 3-4 weeks later and then sacrificed at 5-6 weeks post boost.

### Hemagglutinin Specific ELISA and HAI Analysis of Mouse Sera

Hemagglutinin antigen specific antibodies and HAI titer were measured from immunized mouse serum as previously published (Brewer et al., 2017; Nogales et al., 2018).

### Example 2

Peptides 1 and 2 strongly aggregated when prepared directly in buffer or cell culture media. Screening a series of surfactants to facilitate formation of stable peptide structure to improve handling revealed that the inclusion of 0.12% PLURONIC® F-127 allowed for solubilization and subsequent dilution of Peptides 1 to 4 into cell culture media without precipitation (Khattak, Bhatia, & Roberts, 2005).

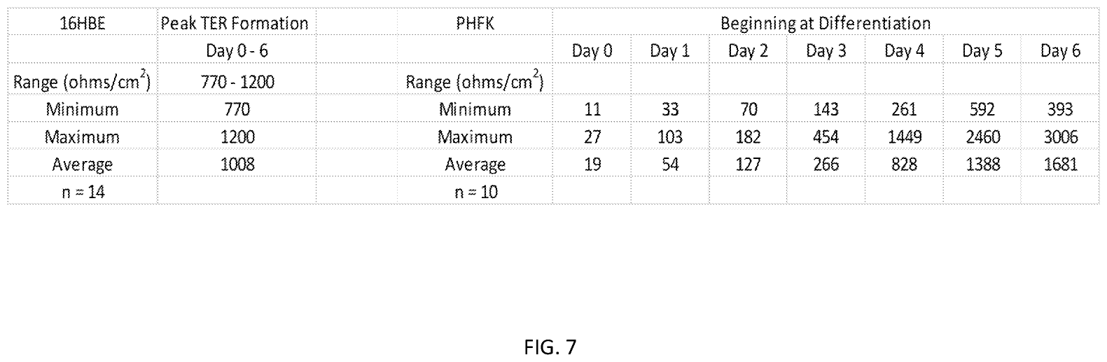

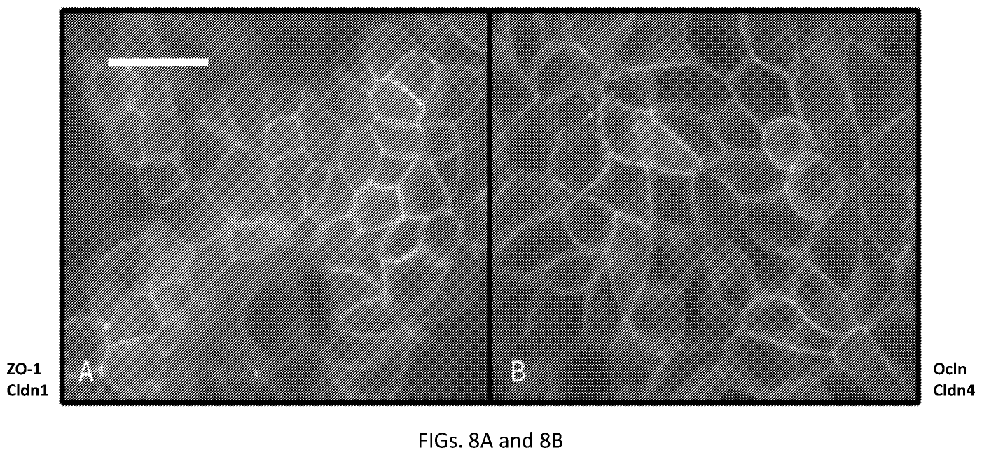

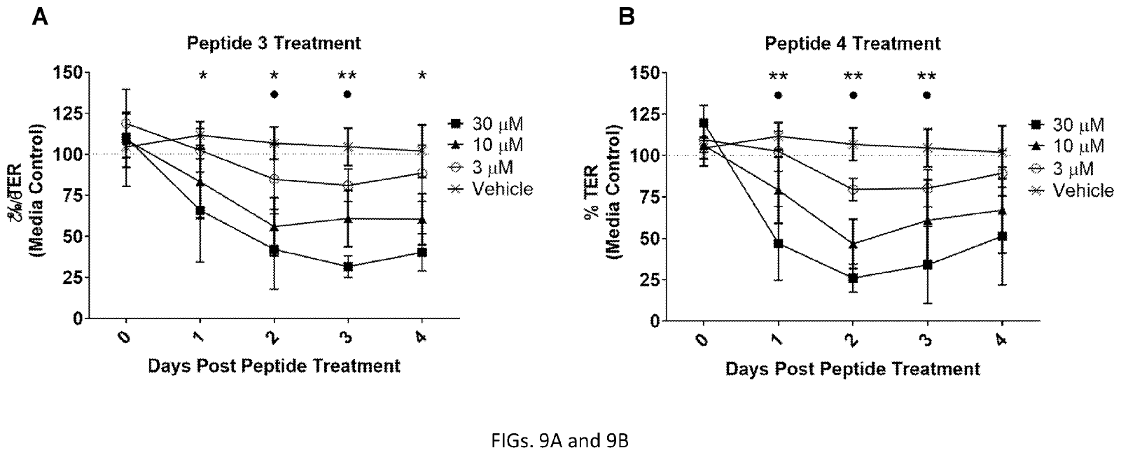

Initial experiments employed the human bronchial epithelial cell line, 16HBE since this cell line is known for its robust TJ formation (Saatian et al., 2013). In the media used for peptide exposure, 16HBE cells achieved a stable TER of 800-1200 Ω*cm² (FIG. 7). Immunofluorescence staining revealed reactivity for the TJ molecules Cldn1and zonula occluders (ZO)-1 at the periphery of epidermal cells with a honeycomb like appearance (FIG. 8). The honeycomb fluorescence pattern of these proteins on the cell surface is a hallmark of a barrier competent TJ (Furuse, Fujita, Hiiragi, Fujimoto, & Tsukita, 1998; Furuse et al., 1993). Following TJ formation, cells were exposed to peptides (0.4 to 50 μm) or vehicle. Peptide 1 or 2 significantly decreased TER (p<0.01, p<0.0001 respectively) (FIG. 2A). The disruption was dose-dependent, with both peptides eliciting minimal disruption at 0.4 μM (2-3%) and increasing reductions at higher concentrations. Peak disruption was observed at day three for Peptide 1 (72% decrease at 50 μM) with even greater disruption observed with Peptide 2 at day two (90% decrease at 50 μM). Peptides 3 and 4 were less effective at disrupting barrier with maximum TER reductions of 68% for Peptide 3 on day three and 74% for Peptide 4 on day two when used at 30 μM (FIG. 9). Importantly, TJ recovered after peptide washout, except at the highest dose (50 μM) and the PLURONIC F-127® surfactant vehicle control did not disrupt TJ. A control peptide tested in the same concentration range ((FKFE)₂; 12 and 96 μM) also did not affect TER values suggesting that TJDPs did not nonspecifically affect barrier integrity (FIG. 9). Using a water soluble tetrazolium-1 (WST-1) assay, inventors observed no loss of viability after treatment of 16HBE with 50 μM of Peptides 1 or 2, the highest concentration tested (FIG. 2B). Inventors observed enhanced TJ penetration of a labeled monoclonal antibody (palivizumab, 150 kDa) in peptide-treated cells (FIG. 2C) (Anderson, Carosone-Link, Yogev, Yi, & Simoes, 2017). Antibody penetration was enhanced 2.5±0.2-fold and 3.2±0.4-fold by 2.4 and 12 μM Peptide 1, respectively. Inventors observed greater permeability after Peptide 2 exposure, with 4.9±0.4-fold and 5.6±0.9-fold enhancement using 2.4 and 12 μM, respectively. The antibody diffusion was measured at 30 min to reduce the likelihood that active transcellular transport was measured.

### Example 3

In order to validate peptide-mediated TJ disruption in skin, PHFK were isolated and propagated from neonatal human foreskins. PHFK begin forming TJ as measured by TER following differentiation in high calcium media (day three, 140-450 ξcm², FIG. 7). To determine if TJ could be perturbed during differentiation, PHFK were treated with Peptide 2 upon addition of high calcium media. Peptide 2 was chosen for all further studies given its robust phenotype in 16HBE. Notably, when PHFK were differentiated (three days post high calcium media), they became refractory to TJ disruption by peptide treatment (data not shown). In contrast, cells treated with Peptide 2 at initiation of differentiation experienced a significant delay in TJ formation for five days (p<0.001) (FIG. 3A). Inventors observed recovery of barrier equivalent to media-treated controls after peptide removal (day three). Modest toxicity was observed at higher concentrations by WST-1 assay (30 μM, p<0.05), but at lower concentrations, where robust inhibition of TJ formation was observed, no appreciable cell death was detected (FIG. 3B).

### Example 4

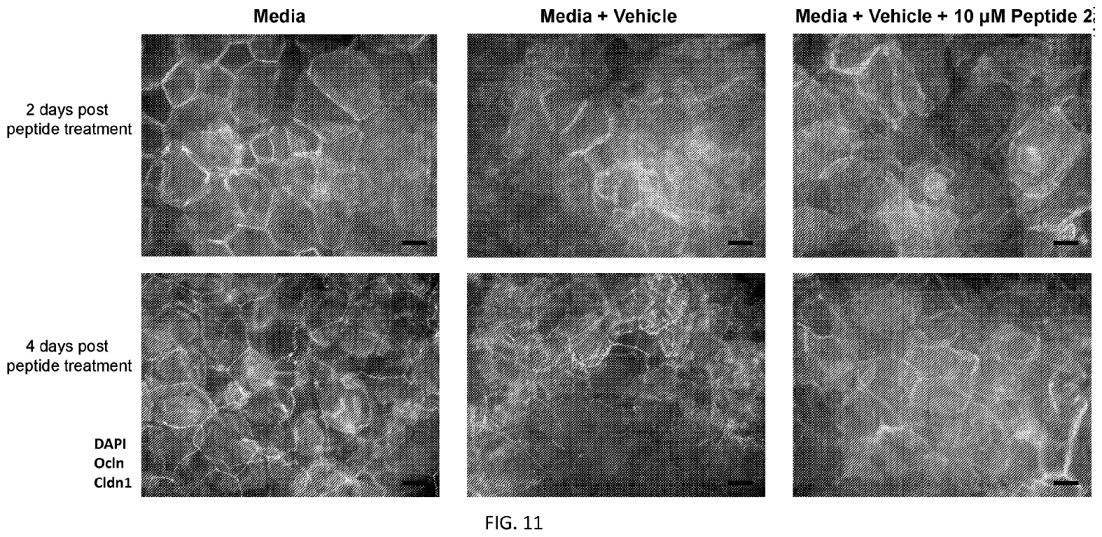

TJ-associated proteins were then examined by immunofluorescence staining of PHFK to determine whether peptide exposure resulted in changes to the appearance or distribution of the key TJ transmembrane proteins, Cldn1and Ocln (FIG. 4A). At two days after peptide treatment, PHFK appeared to have a higher intensity of Cldn1 distributed throughout the monolayer, with some of the staining now observed within the cytoplasm (FIG. 4A top panels, and FIG. 11). At four days post peptide treatment there was a notable lack of honeycomb Ocln staining compared to control cells, consistent with functional changes in TJ (FIG. 4A bottom panels). Four days after Peptide 2 treatment PHFK appeared to have Cldn1distribution similar to control cells at two days demonstrating a peptide-induced delay in TJ organization during barrier formation (FIG. 4A bottom panels). Quantification of DAPI-stained nuclei showed minimal changes in cellular density with mean values of 557, 623, and 527 cells per image for media, vehicle, and Peptide 2 images respectively, supporting the conclusion that peptide treatment does affect barrier function by removal of cells from the monolayer (FIG. 4B). Significant reduction in the magnitude of Ocln staining was observed in cells treated with Peptide 2 at day two and four (FIG. 4B: p<0.05, p<0.01; respectively). Interestingly, greater Cldn1intensity per number of DAPI⁺ PHFK was observed at two and four days after peptide treatment, suggesting aberrant accumulation of TJ-proteins not typically seen during PHFK differentiation.

### Example 5

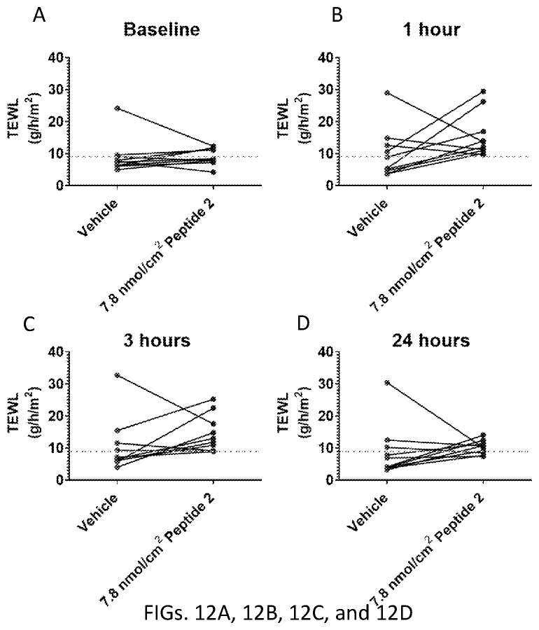

Inventors developed an epicutaneous “patch” delivery system to determine whether the TJDPs described above could impair barrier function in murine skin. To accomplish this, patches containing either Peptide 2 or vehicle were applied to the flanks of animals three days following removal of fur. 18 hours later patches were removed and TEWL was measured at 1, 3, and 24 hours as an indicator of barrier disruption. Patches containing Peptide 2 significantly (p <0.01) increased TEWL at 1 and 3 hours post-patch removal (1.8 and 1.6-fold, respectively) compared to patches containing vehicle, with barrier function returning to baseline after 24 hours (FIG. 5 and FIG. 12).

### Example 6

Building on results described above, inventors investigated whether TJ disruption achieved with the TJDP peptides was sufficient to promote immunological responsiveness to an epicutaneously applied antigen. Influenza A hemagglutinin (HA) was used as a model antigen, in two models: a patch-based prime followed by intramuscular (IM) boost, or an IM prime followed by a patch-based boost. These two models were chosen to simulate a naïve response (patch-prime) to an antigen, or, as occurs with influenza, seasonal boosting of a pre-existing response (patch-boost). No detectable response was measured after a patch-prime containing either vehicle or TJDP with HA (FIGS. 6A-6B). Upon boosting mice with an IM delivery of HA, animals that received a TJDP containing patch during primary exposure to the antigen had significantly (p<0.05) increased HA-specific IgG antibody titers compared to vehicle control, with a mean antibody endpoint titer of 33,000 compared to 6,400 (day 38 post boost; FIGS. 6A-6B). To determine whether patch-based delivery could boost preexisting immunity to an antigen (as is done during annual influenza vaccine campaigns), animals were primed with an IM immunization of influenza and followed that with patch delivery of HA. Animals boosted with HA in a patch containing Peptide 2 had significant increases in the antibody response observed as early as 14 days post-boost. This response was delayed compared to animals that received the boost by IM delivery of antigen, but the same level of antibody titers was achieved by day 28. Additionally, the capability of antibodies elicited in the patch boost experiments to neutralize virus was measured at day 35 by hemagglutination inhibition (HAI) assay, which showed comparable titers to IM control animals (FIGS. 6C-6E). Since all animals began with similar antibody titers this strongly implicates TJ-disruption as a key event promoting robust and protective antigen-specific responses comparable to IM immunization.

### REFERENCES

Anderson, E. J., Carosone-Link, P., Yogev, R., Yi, J., & Simoes, E. A. F. (2017). Effectiveness of Palivizumab in High-risk Infants and Children: A Propensity Score Weighted Regression Analysis. Pediatr Infect Dis J, 36(8), 699-704. doi:10.1097/I NF.0000000000001533 Baumgartner, H. K., Beeman, N., Hodges, R. S., & Neville, M. C. (2011). A D-peptide analog of the second extracellular loop of claudin-3 and -4 leads to mislocalized claudin and cellular apoptosis in mammary epithelial cells. Chem Biol Drug Des, 77(2), 124-136. doi:10.1111/j.1747-0285.2010.01061.x
Beeman, N., Webb, P. G., & Baumgartner, H. K. (2012). Occludin is required for apoptosis when claudin-claudin interactions are disrupted. Cell Death Dis, 3, e273. doi:10.1038/cddis.2012.14
Brewer, M. G., DiPiazza, A., Acklin, J., Feng, C., Sant, A. J., & Dewhurst, S. (2017). Nanoparticles decorated with viral antigens are more immunogenic at low surface density. Vaccine, 35(5), 774-781. doi:10.1016/j.vaccine.2016.12.049
Colegio, O. R., Van Itallie, C., Rahner, C., & Anderson, J. M. (2003). Claudin extracellular domains determine paracellular charge selectivity and resistance but not tight junction fibril architecture. Am J Physiol Cell Physiol, 284(6), C1346-1354. doi:10.1152/ajpce11.00547.2002
Colegio, O. R., Van Itallie, C. M., McCrea, H. J., Rahner, C., & Anderson, J. M. (2002). Claudins create charge-selective channels in the paracellular pathway between epithelial cells. Am J Physiol Cell Physiol, 283(1), C142-147. doi:10.1152/ajpce11.00038.2002
De Benedetto, A., Kubo, A., & Beck, L. A. (2012). Skin barrier disruption: a requirement for allergen sensitization? J Invest Dermatol, 132(3 Pt 2), 949-963. doi:10.1038/jid.2011.435
De Benedetto, A., Rafaels, N. M., McGirt, L. Y., Ivanov, A. I., Georas, S. N., Cheadle, C., et al. (2011). Tight junction defects in patients with atopic dermatitis. J Allergy Clin lmmunol, 127(3), 773-786 e771-777. doi:10.1016/j.jaci.2010.10.018
De Benedetto, A., Slifka, M. K., Rafaels, N. M., Kuo, I. H., Georas, S. N., Boguniewicz, M., et al. (2011). Reductions in claudin-1 may enhance susceptibility to herpes simplex virus 1 infections in atopic dermatitis. J Allergy Clin lmmunol, 128(1), 242-246 e245. doi:10.1016/j.jaci.2011.02.014
Erbelding, E. J., Post, D. J., Stemmy, E. J., Roberts, P. C., Augustine, A. D., Ferguson, S., et al. (2018). A Universal Influenza Vaccine: The Strategic Plan for the National Institute of Allergy and Infectious Diseases. J Infect Dis, 218(3), 347-354. doi:10.1093/infdishiyl03
Furuse, M., Fujita, K., Hiiragi, T., Fujimoto, K., & Tsukita, S. (1998). Claudin-1 and -2: novel integral membrane proteins localizing at tight junctions with no sequence similarity to occludin. J Cell Biol, 141(7), 1539-1550.
Furuse, M., Hata, M., Furuse, K., Yoshida, Y., Haratake, A., Sugitani, Y., et al. (2002). Claudin-based tight junctions are crucial for the mammalian epidermal barrier: a lesson from claudin-1-deficient mice. J Cell Biol, 156(6), 1099-1111. doi:10.1083/jcb.200110122
Furuse, M., Hirase, T., Itoh, M., Nagafuchi, A., Yonemura, S., Tsukita, S., et al. (1993). Occludin—a Novel Integral Membrane-Protein Localizing at Tight Junctions. Journal of Cell Biology, 123(6), 1777-1788. doi:DOI 10.1083/jcb.123.6.1777
Gunzel, D. (2017). Claudins: vital partners in transcellular and paracellular transport coupling. Pflugers Arch, 469(1), 35-44. doi:10.1007/s00424-016-1909-3
Haftek, M., Callejon, S., Sandjeu, Y., Padois, K., Falson, F., Pirot, F., et al. (2011). Compartmentalization of the human stratum corneum by persistent tight junction-like structures. Exp Dermatol, 20(8), 617-621. doi:10.1111/j.1600-0625.2011.01315.x
Ita, K. (2016). Transdermal delivery of vaccines - Recent progress and critical issues. Biomed Pharmacother, 83, 1080-1088. doi:10.1016/j.biopha.2016.08.026
Khattak, S. F., Bhatia, S. R., & Roberts, S. C. (2005). Pluronic F127 as a cell encapsulation material: utilization of membrane-stabilizing agents. Tissue Eng, 11(5-6), 974-983. doi:10.1089/ten.2005.11.974
Kubo, A., Nagao, K., & Amagai, M. (2012). Epidermal barrier dysfunction and cutaneous sensitization in atopic diseases. J Clin Invest, 122(2), 440-447. doi:10.1172/JC157416
Leone, M., Monkare, J., Bouwstra, J. A., & Kersten, G. (2017). Dissolving Microneedle Patches for Dermal Vaccination. Pharm Res, 34(11), 2223-2240. doi:10.1007/s11095-017-2223-2
Levin, Y., Kochba, E., & Kenney, R. (2014). Clinical evaluation of a novel microneedle device for intradermal delivery of an influenza vaccine: are all delivery methods the same? Vaccine, 32(34), 4249-4252. doi:10.1016/j.vaccine.2014.03.024
Liu, L., Fuhlbrigge, R. C., Karibian, K., Tian, T., & Kupper, T. S. (2006). Dynamic programming of CD8+ T cell trafficking after live viral immunization. Immunity, 25(3), 511-520. doi:10.1016/j.immuni.2006.06.019
Mrsny, R. J., Brown, G. T., Gerner-Smidt, K., Buret, A. G., Meddings, J. B., Quan, C., et al. (2008). A key claudin extracellular loop domain is critical for epithelial barrier integrity. Am J Pathol, 172(4), 905-915. doi:10.2353/ajpath.2008.070698
Nachbagauer, R., Liu, W. C., Choi, A., Wohlbold, T. J., Atlas, T., Rajendran, M., et al. (2017). A universal influenza virus vaccine candidate confers protection against pandemic H1N1 infection in preclinical ferret studies. NPJ Vaccines, 2, 26. doi:10.1038/s41541-017-0026-4
Nogales, A., Piepenbrink, M. S., Wang, J., Ortega, S., Basu, M., Fucile, C. F., et al. (2018). A Highly Potent and Broadly Neutralizing H1 Influenza-Specific Human Monoclonal Antibody. Sci Rep, 8(1), 4374. doi:10.1038/s41598-018-22307-8
O'Neill, C. A., & Garrod, D. (2011). Tight junction proteins and the epidermis. Exp Dermatol, 20(2), 88-91. doi:10.1111/j.1600-0625.2010.01206.x
Plotkin, S. A. (2010). Correlates of protection induced by vaccination. Clin Vaccine Immunol, 17(7), 1055-1065. doi:10.1128/CVI.00131-10
Poumay, Y., Roland, I. H., Leclercq-Smekens, M., & Leloup, R. (1994). Basal detachment of the epidermis using dispase: tissue spatial organization and fate of integrin alpha 6 beta 4 and hemidesmosomes. J Invest Dermatol, 102(1), 111-117.
Roussel, A. J., Bruet, V., Marsella, R., Knol, A. C., & Bourdeau, P. J. (2015). Tight junction proteins in the canine epidermis: a pilot study on their distribution in normal and in high IgE-producing canines. Can J Vet Res, 79(1), 46-51.
Saatian, B., Rezaee, F., Desando, S., Emo, J., Chapman, T., Knowlden, S., et al. (2013). Interleukin-4 and interleukin-13 cause barrier dysfunction in human airway epithelial cells. Tissue Barriers, 1(2), e24333. doi:10.4161/tisb.24333
Schmidt, S. T., Khadke, S., Korsholm, K. S., Perrie, Y., Rades, T., Andersen, P., et al. (2016). The administration route is decisive for the ability of the vaccine adjuvant CAF09 to induce antigen-specific CD8(+) T-cell responses: The immunological consequences of the biodistribution profile. J Control Release, 239, 107-117. doi:10.1016/j.jconre1.2016.08.034
Sugawara, T., Iwamoto, N., Akashi, M., Kojima, T., Hisatsune, J., Sugai, M., et al. (2013). Tight junction dysfunction in the stratum granulosum leads to aberrant stratum corneum barrier function in claudin-1-deficient mice. J Dermatol Sci, 70(1), 12-18. doi:10.1016/j.jdermsci.2013.01.002
Todorova, B., Adam, L., Culina, S., Boisgard, R., Martinon, F., Cosma, A., et al. (2017). Electroporation as a vaccine delivery system and a natural adjuvant to intradermal administration of plasmid DNA in macaques. Sci Rep, 7(1), 4122. doi:10.1038/s41598-017-04547-2
Tokumasu, R., Tamura, A., & Tsukita, S. (2017). Time- and dose-dependent claudin contribution to biological functions: Lessons from claudin-1 in skin. Tissue Barriers, 5(3), e1336194. doi:10.1080/21688370.2017.1336194
Wong, V., & Gumbiner, B. M. (1997). A synthetic peptide corresponding to the extracellular domain of occludin perturbs the tight junction permeability barrier. J Cell Biol, 136(2), 399-409.
Yoshida, K., Yokouchi, M., Nagao, K., Ishii, K., Amagai, M., & Kubo, A. (2013). Functional tight junction barrier localizes in the second layer of the stratum granulosum of human epidermis. J Dermatol Sci, 71(2), 89-99. doi:10.1016/j.jdermsci.2013.04.021
Zaric, M., Becker, P. D., Hervouet, C., Kalcheva, P., Ibarzo Yus, B., Cocita, C., et al. (2017). Long-lived tissue resident HIV-1 specific memory CD8(+) T cells are generated by skin immunization with live virus vectored microneedle arrays. J Control Release, 268, 166-175. doi:10.1016/j.jconre1.2017.10.026
Zhou, H., Thompson, W. W., Viboud, C. G., Ringholz, C. M., Cheng, P. Y., Steiner, C., et al. (2012). Hospitalizations associated with influenza and respiratory syncytial virus in the United States, 1993-2008. Clin Infect Dis, 54(10), 1427-1436. doi:10.1093/cid/cis211
Zwanziger, D., Hackel, D., Staat, C., Bocker, A., Brack, A., Beyermann, M., et al. (2012). A peptidomimetic tight junction modulator to improve regional analgesia. Mol Pharm, 9(6), 1785-1794. doi:10.1021/mp3000937
Zwanziger, D., Staat, C., Andjelkovic, A. V., & Blasig, I. E. (2012). Claudin-derived peptides are internalized via specific endocytosis pathways. Ann N Y Acad Sci, 1257, 29-37. doi:10.1111/j.1749-6632.2012.06567.x

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. An isolated polypeptide comprising a sequence that is at least 80% identical to SEQ ID NO: 3 or 4.

2. The isolated polypeptide of claim 1, wherein the polypeptide comprises the sequence of SEQ ID NO: 3 or 4.

3. A transepithelial delivery system or transepithelial delivery composition comprising (i) the polypeptide of claim 1 and (ii) a pharmaceutically acceptable carrier.

4. The transepithelial delivery system or transepithelial delivery composition of claim 3, further comprising (iii) an active agent.

5. A therapeutic composition comprising the transepithelial delivery composition of claim 4, wherein the active agent comprises an effective amount of a therapeutic agent.

6. The therapeutic composition of claim 5, wherein the therapeutic agent comprises a small molecule, a biologic, a nanoparticle, a protein, a nucleic acid, or a combination thereof

7. An immunogenic composition comprising the transepithelial delivery composition of claim 4, wherein the active agent comprises an effective amount of an antigenic agent.

8. The immunogenic composition of claim 7, wherein the antigenic agent comprises one or more selected from the group consisting of a polysaccharide, a lipid, a protein, a nucleic acid, a small molecule, and a toxin, or an epitope thereof

9. The immunogenic composition of claim 7, wherein the antigenic agent comprises an antigen of a pathogen or an epitope thereof

10. The immunogenic composition of claim 9, wherein the pathogen is a virus, a bacterium, a fungus, or a parasite.

11. The immunogenic composition of claim 10, wherein the virus is selected from the group consisting of a picornavirus, a togovirus, a coronavirus, an arenavirus, a bunyavirus, a rhabdovirus, an orthomyxovirus, a paramyxovirus, a reovirus, a parvovirus, a papovovirus, an adenovirus, a herpesvirus, a varicella-zoster virus, and an RNA tumor virus.

12. The immunogenic composition of claim 8, wherein the virus is an influenza virus.

13. The immunogenic composition of claim 7, wherein the antigenic agent comprises a tumor antigen or an epitope thereof

14. The immunogenic composition of claim 7, wherein the antigenic agent comprises an allergen or an epitope thereof

15. The transepithelial delivery system, transepithelial delivery composition therapeutic composition, or immunogenic composition of claim 3, being in the form of a transdermal patch.

16. A method of producing antibodies that recognize an antigen, or eliciting an antigen-specific immune response, in a subject, comprising administering to the subject the immunogenic composition of claim 7.

17. An isolated nucleic acid comprising a sequence encoding the polypeptide of any of claim 1.

18. An expression vector comprising a nucleic acid of claim 17.

19. A host cell comprising a nucleic acid of claim 17.

20. A method of producing a polypeptide, comprising culturing the host cell of claim 19 in a medium under conditions permitting expression of a polypeptide encoded by the nucleic acid, and purifying the polypeptide from the cultured cell or the medium of the cell.


SEQUENCE LISTING (4 sequences)

SEQ ID NO: 1 - protein, 29 aa, Artificial
  Synthetic
SSVSQSTGQIQSKVFDSLLNLSSTLQATR

SEQ ID NO: 2 - protein, 29 aa, Artificial
  Synthetic
SILTGVSTLDQSLKQLSNFSQAVSTQSSR

SEQ ID NO: 3 - protein, 29 aa, Artificial
  Synthetic
SILTGVSTLGNTLGQLTNFSNAVSTQTSR

SEQ ID NO: 4 - protein, 29 aa, Artificial
  Synthetic
SILTGVSTLDNTLGQLTNFSNAVSTQTSR